(19) United States
(12) Patent Application Publication
(10) Pub. No.: US 2004/0210400 A1
(43) Pub. Date: Oct. 21, 2004

(54) Analysis methods for individual genotyping

(75) Inventor: Karel Konvicka, Palo Alto, CA (US)

Correspondence Address: PERLEGEN SCIENCES, INC., LEGAL DEPARTMENT, 2021 STIERLIN COURT, MOUNTAIN VIEW, CA, 94043, US

(73) Assignee: Perlegen Sciences, Inc., Mountain View, CA (US)

(21) Appl. No.: 10/786,475

(22) Filed: Feb. 24, 2004

Related U.S. Application Data
(63) Continuation-in-part of application No. 10/351,973, filed on Jan. 27, 2003.

(51) Int. Cl. G06F 19/00; G01N 33/48

(52) U.S. Cl. 702/20

(57) ABSTRACT

A method and data processing apparatus for determining the genotype of at least one individual from a genetic marker using at least one measure of the amount of an allele of the genetic marker that the individual are described. The method assigns the measure of the amount of an allele to a group, a genotype is then assigned to the group and the individual is determined to have the genotype assigned to the group.

### CROSS-REFERENCE TO RELATED APPLICATIONS

The present application is a continuation-in-part of U.S. patent application Ser. No. 10/351,973, filed Jan. 27, 2003, entitled “Apparatus and Methods for Determining Individual Genotypes”, the disclosure of which is specifically incorporated herein by reference in its entirety.

### BACKGROUND OF THE INVENTION

The sequence of bases in DNA encodes genetic information of an organism. As is well known the differences at a genetic level between different species can be relatively small, while still resulting in significant differences in the phenotype of the species. For example the DNA sequences of humans and other primates are very similar although the phenotypic characteristics of these organisms differ significantly. Further, there can be significant differences in the phenotypes of different groups within the same species, e.g. racial groups such as Mestizos and Caucasians, wherein genetically the individuals are very similar but there are still some gross phenotypic differences, such as height, between the groups. Some variations in the genetic sequences of individuals lead to phenotypic differences such as resistance or susceptibility to diseases or illnesses, or other physical characteristics or conditions.

In investigating or studying the genetic basis for variations in phenotypes between organisms, it can be useful to be able to determine the specific genetic sequence that an individual has at a particular position, group of positions, region or group of regions in their actual genome. As the genome for different individuals of the same species, or indeed for certain different species, tend to be very similar, such studies can focus on identifying and investigating the properties of the differences in the genetic sequences, rather than comparing genomes as wholes, which is not rapidly practicable. Therefore, it can be useful to be able to determine the genotype of a specific individual organism for these areas of variation in the genetic sequence, in order to try and correlate the variations at the genetic level of individuals with variations in the same individuals' phenotypic characteristics.

The present invention therefore relates to methods, apparatus and processes for determining the genotype of an individual organism or several individual organisms for one or more genetic markers in the individuals' genomes.

### SUMMARY OF THE INVENTION

To achieve the foregoing, and in accordance with the purpose of the present invention, a method, apparatus and computer program code for individual genotyping is disclosed.

A method for determining the genotype of at least one individual from a genetic marker is provided. The method can use at least one measure of the amount of an allele of the genetic marker that the individual has. The measure of the amount of an allele can be assigned to a group. A genotype can be assigned to the group based on a property of the group. In this way, the individual is determined to have the genotype assigned to the group.

In an embodiment, a probability clustering process can be used to assign the amount of an allele to a group. In another embodiment, a distance-based clustering process can be used. In another embodiment, both a probability clustering process and a distance-based clustering process are used. Favorably, the probability clustering process involves an expectation maximization algorithm. Similarly, the distance-based clustering process can involve a K-means algorithm.

According to another aspect of the invention, there is provided a method for determining the genotypes of a plurality of individuals. Respective measures of the relative allele amount for a SNP position for each individual can be used. The measures of the relative allele amount can be assigned to a group using an expectation maximization process. A genotype can be assigned to each group identified by the expectation maximization process. In this way the genotype of each person can be determined. Optionally, the reliability of determination of the genotype can be assessed.

The invention also provides data processing apparatus for determining the genotype of an individual from a genetic marker using a measure of the amount of an allele of the genetic marker that the individual has. The apparatus can includes a data processor and a storage device holding computer readable code in communication with the data processor. The computer readable code can include computer code which can execute a probability clustering that assigns the measure of the amount of an allele and computer code which queries a database to assign genotype to a cluster. Computer code can also be provided to assign a genotype to the group based on a property of the group. Computer code can also determine the individual as having the genotype assigned to the group.

The invention also provides a computer readable medium holding computer readable code for determining the genotype of an individual from a genetic marker using a measure of the amount of an allele of the genetic marker that the individual has. The computer code can carry out a number of processes, including assigning the measure of the amount of an allele to a group. The computer code can execute at least one of a probability clustering process or a distance-based clustering process to assign the measure to a group. Computer code can also assign a genotype to the group based on a property of the group. The computer code can determine the individual to have the genotype assigned to the group.

According to further aspects of the invention, there is also provided a method for calibrating a range of values of measures of the presence of an allele at a genetic marker. The method can include measuring the value of a measure of the presence of an allele for a group of individuals. Preferably there are sufficient individuals to allow groups of individuals corresponding to all genotypes to be reliably determined. The boundaries between distributions can be determined. Preferably the boundaries are determined by fitting the distribution of values for each group of individuals. A range of values of the measure of the presence of an allele can be associated with each genotype. At least one boundary of each range can be determined by the intersect of the distributions of adjacent groups.

According to a yet further aspect of the invention, there is provided a method for carrying out a clustering process, e.g., a probability clustering process. The method involves establishing a dissimilarity in groups of values of measures of the presence of an allele at a genetic marker. The method can include measuring the values of the presence of an allele at the genetic marker at least twice for the same group of individuals. The values of the presence of an allele can be assigned to a number of groups using, e.g., a probability clustering algorithm and/or a distance-based clustering algorithm. The number of groups to which to assign the values can be determined using a cut off separation of assigned groups for the independent measurements. A genotype can be assigned to each group for a cut off value and for each of the independent measurements. The number of inconsistent genotype assignments can be minimized for the same individual as a function of the cut off.

### BRIEF DESCRIPTION OF THE DRAWINGS

The invention, together with further advantages thereof, may best be understood by reference to the following description taken in conjunction with the accompanying drawings in which:

FIG. 1 shows a flow chart illustrating a general experimental method for genotyping individual organisms;

FIG. 2 shows a flow chart illustrating various data processing operations carried out in calculating a measure of the amount of an allele present;

FIGS. 3A, 3B, 3C and 3D respectively show probe tiling patterns for an oligonucleotide probe array for use in the method illustrated by FIG. 1;

FIG. 4 shows a schematic diagram illustrating the architecture of a software process for implementing the method of the invention;

FIGS. 5A and 5B show flow charts illustrating alternative embodiments of the processes of the invention in greater detail.

FIG. 6 shows a database schema for a database used by the invention;

FIG. 7 shows a flow chart illustrating processes carried out in step 420 of FIG. 5 in greater detail;

FIG. 8 shows a flow chart illustrating processes carried out in step 422 of FIG. 5 in greater detail;

FIG. 9 shows a flow chart illustrating a process for determining a cluster separation cut off value as used by the method of the invention;

FIG. 10 shows a flow chart illustrating step 818 of FIG. 9 in greater detail;

FIG. 11 shows a flow chart illustrating a calibration process used by the method of the invention;

FIG. 12 shows graphical representations of distributions of P′ mean values for SNPs that have all three distributions used by the calibration process illustrated by FIG. 11; and

FIGS. 13A and 13B respectively show a computer system and a schematic diagram of data processing apparatus of the computer system according to an aspect of the invention.

In the Figures, like reference numerals refer to like components and elements

### DETAILED DESCRIPTION

The present invention relates to methods, apparatus and computer programs for use in determining the genotype of an individual or individuals. A genetic marker, which corresponds to a region of interest in the genome of an individual is investigated and the particular allele or alleles of the genetic marker present for the individual is used in assigning a genotype to the individual for that genetic marker. The individual genotypes for a plurality of individuals can be determined. The genotype for a plurality of genetic markers for an individual or a group of individuals can be determined. The genotypes of the individuals can be used in investigating any association between the genetic markers of the individuals and phenotypic characteristics of the individuals.

Any suitable genetic marker can be used. A genetic marker can be located in any nucleotide sequence in the individual's DNA. For example, the genetic marker can be a part of a gene sequence, an intron, an exon, a sequence corresponding to a tRNA, mRNA, rRNA, or other RNA, a codon, a polymorphic sequence or any ad hoc nucleotide sequence. The genetic marker may be at a single nucleotide position or may span multiple nucleotide positions. The genetic marker or markers are preferably sequences which tend to, are believed to, or are known to vary between individuals or species and as such are likely to be associated with variations in phenotypes. It can be that a particular phenotype depends on the presence of a particular combination of standard sequences and variable sequences and so the genetic marker can also be a combination of nucleotide sequences. Further, the genetic marker may be a nucleotide sequence which itself does not directly cause the manifestation of the phenotype of interest, but which may still be associated with the variation in phenotype by virtue of a genetic linkage to a nucleotide sequence that is directly involved in the manifestation of the phenotype of interest.

A detailed description of an embodiment of the invention will be presented using bi-allelic single nucleotide polymorphisms (herein after “SNPs”) as an example of a genetic marker. However, this is by way of example only and the invention is not considered to be limited to this particular genetic marker nor only to bi-allelic markers. The application of the general principles enumerated hereinafter to other types of genetic markers and different numbers of alleles will be apparent to a person of ordinary skill in the art in light of the teaching herein.

Further, an embodiment of the invention will be described with reference to genotyping SNPs in human DNA. However, the invention is not limited to such applications, and can be used to characterize multi-allelic genetic markers in nucleic acid sequences derived from any organism, such as an animal, human, insect, bacterium, etc. with the proviso that the Hardy-Weinberg equilibrium is typically used in conjunction with biallelic genetic markers. In any reference to DNA herein such reference may include derivatives of DNA such as amplicons, RNA transcripts, cDNAs, nucleic acid mimetics, and the like.

A SNP (“single nucleotide polymorphism”) is a variation in the allele at a particular nucleotide position established by comparison of the allele at that position for an individual with the allele at that position in the human genome sequence (HGS), or a version thereof. A nucleotide position can be identified by its absolute position in a sequence or by the sequence of bases in the locale of the position, as it is common for the ‘same position’ in a DNA sequence to be in different actual positions in the DNA sequences of different HGS versions. Irrespective of how the position is identified or defined, at a SNP position in a DNA sequence, a person will in general have a particular allele, i.e. the base C, G., T or A, present at the SNP position (absent any nucleotide deletion mutations). The allele corresponding to the HGS or other reference sequence, is referred to as the reference allele. In general SNPs are bi-allelic and the other allele possible at a SNP position (i.e., not the same as the reference allele) is referred to as the alternate allele.

As humans are diploid, there are three possible genotypes for a biallelic SNP. If the alleles at the SNP position on each of the parental chromosomes are both the reference allele, then the genotype of the individual for that SNP position is homozygous reference. Similarly, if the alleles at the SNP position on each of the parental chromosomes are both the alternate allele, then the genotype of the individual for that SNP position is homozygous alternate. If the allele at the SNP position on a one of the parental chromosomes is the reference allele and the allele at the SNP position on the other of the parental chromosomes is the alternate allele, then the genotype is heterozygous. For haploid organisms, and a biallelic marker, there will be the genotypes reference and alternate only. Likewise, in diploid organisms some of the genetic markers may be in a haploid state. For example, typically human males have only one X and one Y chromosome, so the genetic markers present on these chromosomes are in a haploid state and thus can have only the genotypes of reference and alternate. Therefore, the genotype of an individual for a genetic marker can be assigned by determining what alleles are present in the individual's DNA and by obtaining a measure of the relative allele presence, i.e. how much of one allele is present in the DNA of an individual compared to any other possible alleles.

As explained above, SNPs are defined with reference to the HGS, and so correspond to a variation away from the reference HGS sequence. However, the HGS is not itself a ‘correct’ genetic sequence, but rather is a composite from different individuals and so some of the sequence will be specific to those individuals and not global. Similarly, each person may have nucleotide positions which differ in a unique way from the HGS sequence. These also are a reflection of the individuals and may be considered a ‘rare’ SNP of interest if they do not also occur in a significant number of additional individuals. Further, the HGS sequence may itself contain ‘rare’ SNPs of interest; that is, it may contain a polymorphism that occurs very rarely in populations of individuals. However, when a significant number of a population of individuals all have an allele at a nucleotide position which is the same for those people, but different to that of the HGS (alternate allele), and a significant number of individuals in the same population have the HGS allele (reference allele), then this can be considered a ‘common’ SNP of interest as there is some likelihood that it is somehow related to a phenotype ‘common’ to the subset of the population carrying the alternate allele (or the subset of the population carrying the reference allele). Typically, ‘common’ SNPs occur commonly in at least one population of individuals; for example, the population may have approximately 5%-95% of a particular allele, or about 15%-80%, or about 25%-75%, or about 40%-60% of a particular allele. Typically, SNPs that are common to a relatively large number of people are much less frequent in the genome than SNPs that are unique to only a few people. Unless the context indicates otherwise, the following will refer to the both ‘common’ SNPs and ‘rare’ SNPs.

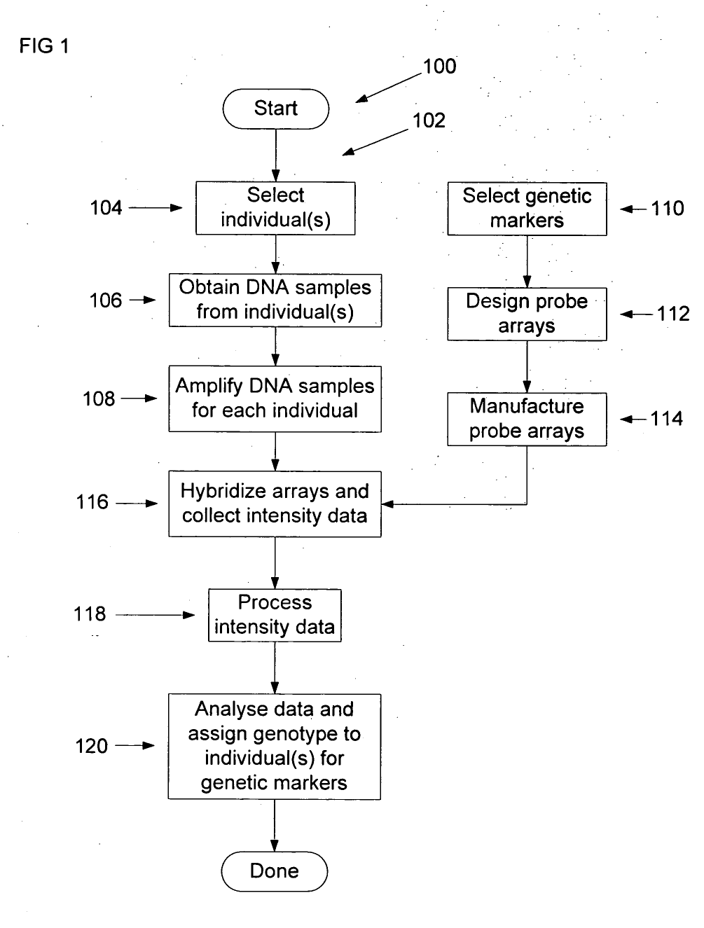

FIG. 1 shows a flow chart 100 illustrating a general experimental method 102 by which data can be obtained for analysis. The flow chart initially has two parallel limbs of flow. Although shown as two parallel limbs of flow for the purpose of illustration, it will be apparent to one of skill that in practice these two limbs need not be performed in parallel but may instead be performed independent of one another, for example, sequentially. In a first limb, an individual or individuals that are to be genotyped are selected 104. The individuals may be selected as members of a case group, exhibiting a phenotype of interest, e.g. a physical characteristic, disease, condition or response to a drug or other medicament—essentially any phenotype wherein a genetic component is causative of the phenotype. The individuals may be selected to be members of a control group believed not to exhibit the phenotype. The nature of the individuals or groups of individuals selected will depend on the purpose of the experimental study or investigation.

In a next step, DNA-containing samples, e.g. blood, skin cells, or saliva, are obtained from the individuals 106 according to any well-known method. The DNA samples for each individual are then separately amplified using PCR and suitable primer pairs in order to provide suitable amplicons for subsequent use with an oligonucleotide probe array, which may also be referred to as a nucleic acid probe array or a DNA probe array.

In a parallel limb, a particular genetic marker or markers are selected for investigation 110. SNPs may be chosen from those publicly available, for example, from dbSNP (http://www.ncbi.nlm.nih.gov/SNP/), the Human SNP Database (http://www.broad.mit.edu/snp/human/), or the SNP Consortium (http://snp.cshi.org/); or may be identified independently by methods known to those of skill in the art (e.g., sequencing of genomic DNA, etc.). Approximately 1.7 million SNP positions have been identified to date, and the reference and alternate alleles determined experimentally. Step 110 can include selecting a particular group of SNPs for investigation. For example, step 110 can include selecting those SNPs on the same chromosome, Alternatively, step 110 can include selecting a number of SNPs at random, or a number of SNPs believed to be involved or associated with a disease or condition mechanism. Alternatively, step 110 can include selecting SNPs from across a genome, or selecting all approximately 1.7 million SNPs. Once a SNP or group of SNPs has been selected, the probes for an oligonucleotide probe array that may be used to detect the selected SNPs in a DNA sample are designed. Suitable tilings of probes for the array will be described in greater detail below. The DNA probe arrays are then manufactured according to the design in step 114.

DNA probe array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been placed. Each of these wafers can hold, for example, approximately 60 million DNA probes that are used to recognize longer sample DNA sequences. The recognition of sample DNA by the set of DNA probes on the glass wafer takes place through the mechanism of DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of DNA is present or not in the sample DNA, the known sequence of DNA being the complement to the sequence of the DNA probes on the array.

The use of DNA probe arrays to obtain genetic information involves the following general steps: design and manufacture of DNA probe array wafers, preparation of the sample, hybridization of target DNA to the array, detection of hybridization events and data analysis to determine whether sequences complementary to those on the array are present in the sample. Preferred wafers are manufactured using a process adapted from semiconductor manufacturing to achieve cost effectiveness and high quality, and are available from Affymetrix, Inc of California. Tiling oligonucleotides on arrays is further described in Chee, et al., Science 274:610 (1996), which is incorporated by reference herein. For details on the use of DNA chips for the detection of, for example, SNPs, see U.S. Pat. No. 6,300,063 issued to Lipshultz, et al., and U.S. Pat. No. 5,837,832 to Chee, et al., HuSNP Mapping Assay, reagent kit and user manual, Affymetrix Part No. 90094 (Affymetrix, Santa Clara, Calif.), all incorporated by reference herein for every purpose.

Probe arrays can be manufactured by light-directed chemical synthesis process, which combines solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays can be synthesized simultaneously on a large glass wafer. This parallel process enhances reproducibility and helps achieve economies of scale.

Once fabricated, DNA probe arrays can be used to obtain genetic information. In general, DNA samples are tagged with a detectable marker (i.e., “labeled”) to facilitate detection of hybridization of the samples to a DNA probe array. In one embodiment, the DNA samples are tagged with a fluorescent reporter group by standard biochemical methods. The labeled samples are incubated with a DNA probe array, and segments of the samples bind, or hybridize, with complementary sequences on the DNA probe array. The DNA probe array is then scanned and the patterns of hybridization are detected by emission of light from the fluorescent reporter groups. Because the identity (e.g., nucleotide sequence) and position of each probe on the DNA probe array is known, the nature of the DNA sequences in the sample applied to the DNA probe array can be determined. When these arrays are used for genotyping experiments, they may be referred to as genotyping arrays.

Once fabricated 114, the arrays are ready for hybridization 116. As described above, the nucleic acid sample to be analyzed is isolated, amplified and labeled with a detectable marker (e.g., a fluorescent reporter group). The labeled nucleic acid sample is then incubated with the array using a fluidics station and hybridization oven. After the hybridization reaction is complete, the array is inserted into a scanner, where patterns of hybridization are analyzed based on the detection of the detectable marker labeling the nucleic acid sample. In some embodiments, these data may be referred to as “probe intensities”. In certain embodiments, the hybridization data (or probe intensities) are collected as light emitted from the fluorescent reporter groups already incorporated into the labeled nucleic acid, which is now bound to the probe array. Probes that most clearly match (are most complementary to) the labeled nucleic acid produce stronger (higher intensity) signals than those that have mismatches. Since the sequence and position of each probe on the array are known, by complementarity, the identity of the nucleic acid sample applied to the probe array can be identified.

In one embodiment, different DNA samples from a single individual are differentially labeled and hybridized with a single set (i.e., one or more) of the designed genotyping arrays. For example, a DNA sample from a tumor from an individual might be labeled differently than a DNA sample from normal tissue from the same individual, with both samples being applied to the same array or set of arrays. Alternatively, DNA samples from two individuals may be differentially labeled (i.e., a first individual labeled with one label and the other labeled with a different label) and hybridized to a single set of the designed genotyping arrays. In this way two sets of data can be obtained from a single physical array. Labels that may be used include, but are not limited to, cychrome, fluorescein, Alexa-488, radioisotopes or biotin (later stained with phycoerythrin-streptavidin after hybridization). Two-color labeling is described in U.S. Pat. No. 6,342,355, incorporated herein by reference in its entirety. Each array may be scanned such that the signal from both labels is detected simultaneously, or may be scanned twice to detect each signal separately. It has been found that measured intensities for cychrome and fluorescein labeled samples can have a non-linear relationship. One origin of this effect is believed to be owing to the saturation of sample molecules with cychrome. Without wishing to be bound by theory, it is believed that adjacent cychrome on the surface of the labeled molecules may interfere and reduce the amount of light emitted. Therefore, in one embodiment, only a single labeling (or a single staining step) with cychrome is used, rather than two stains. Alternatively, a reduced amount of cychrome effective to reduce the non-linearity in detected intensity the can be used in the staining. In certain embodiments, the genetic materials are labeled with a detectable marker prior to application to an array; in certain embodiments, the genetic materials are further stained with a detectable marker after application to the array.

Intensity data is collected by the scanner for all the SNP positions and for each of the individuals separately. (One preferred embodiment of a scanner is disclosed in U.S. Pat. No. 6,586,750, filed Aug. 3, 2001, entitled “High Performance Substrate Scanning”, incorporated herein by reference in its entirety.) Therefore, the measured intensities are a measure indicative of the amount of a particular allele present at the SNP positions for a single individual. The intensity data is then processed 118.

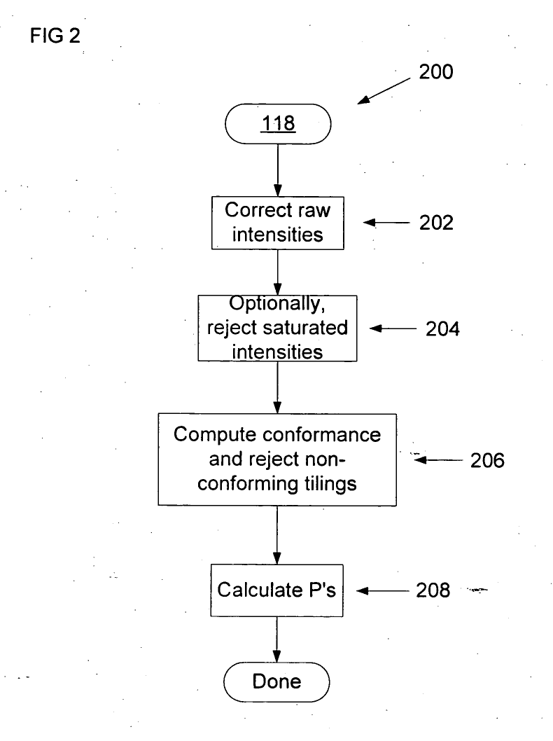

FIG. 2 shows a flow chart 200 illustrating a number of data processing and correcting operations, corresponding to step 118 in FIG. 1, which can be carried out on the intensity measurements obtained from the probe array and before carrying out an analysis to assign genotypes to each SNP 120, as will be described further below.

In a first step, the raw intensity data measurements are corrected 202. A number of intensity correction routines can be carried out on the raw intensity data. A background intensity can be subtracted from all of the intensity measurements. In one embodiment, the value of the background intensity is set at the intensity of the thousandth lowest intensity. That is a histogram of probe cell intensity is determined and the thousandth ranked dimmest probe cell is identified. The measured intensity for that probe cell is subtracted from all the intensities as a background correction. Probe cells that have a negative intensity measure after subtracting a background correction may be discarded from further evaluation.

In an embodiment in which the case and control samples are differently marked or labeled, step 202 may optionally correct for differences in detected intensity which are marker-dependent. For example, it has been found that the measured intensities for labeling with cychrome and fluorescein do not have a linear relationship. A quadratic fit to measured cychrome and fluorescein intensity data can be carried out to provide a quadratic correction to the cychrome intensity data. The measured cychrome intensity data can then be subject to this quadratic correction. At high cychrome intensities a parabolic correction function can be used. In some embodiments, the correction can be scanner-dependent and so different correction data may be used for intensity data collected from different scanners. The non-label-related background corrections described above may be carried out before or after any label-related corrections to the data have been carried out. In some instances, the background correction can be carried out on a SNP by SNP basis.

In a next data processing step 204, saturated probe cells optionally can be discarded from further evaluation. If the photomultiplier tube of the scanner when measuring the probe cell saturates (i.e., there is more signal than the tube can accurately measure), then the measured intensity is not proportional to the actual amount of sample present at the probe cell. Therefore, data collected from the scanner during measurement of the raw intensity data, or the intensity data having a maximum saturated value, is used to determine if the intensity is in fact a saturated intensity value in which case the intensity can be discarded from further evaluation.

In a next data processing step 206, a conformance value, C, is calculated 206 for the remaining intensity data, which provides an indication of the reliability of data, and can be used to remove unreliable data from the data used for subsequent analysis. In general terms, the conformance calculation step 206 involves computing the conformance for every SNP tiled on each of the arrays used in the experiments. The conformance calculation uses the non-discarded and corrected measured light intensity data. The conformance calculation process will be described in greater detail below. Conformance values for all the SNPs are thresholded and the data for those SNPs having a conformance less than the threshold value are rejected from the data set used in the calculation of P′ values. In an embodiment, the conformance threshold can be greater than approximately 0.8 and can be approximately 0.9 or greater.

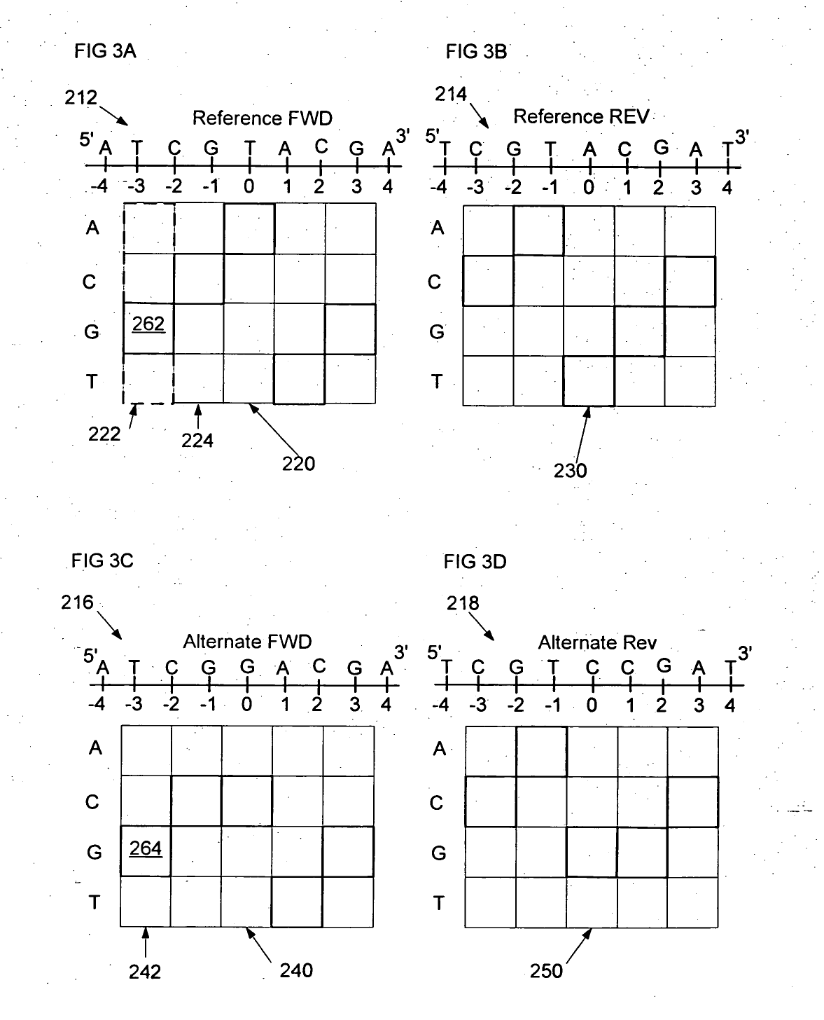

In the illustrated embodiment of the invention, for each particular SNP position of interest, or “target” SNP to be interrogated, a set of eighty probes or “a tiling” of probes is provided on an array or distributed over one or more physical arrays. FIGS. 3A, 3B, 3C and 3D respectively show probe tilings 220, 230, 240, 250 of an array associated with the target SNP position. The target-SNP position has associated with it a forward reference sequence 212. The numbering of the nucleotides along the sequence indicates the position of a nucleotide relative to the SNP position which is indicated by position 0. The target SNP also has a reverse reference sequence 214 associated with it, which is complementary to the forward reference sequence. The allele at the SNP position for the forward reference sequence is T.

The target SNP also has a forward alternate sequence 216 associated with it, including the alternate allele, G, at the SNP position, and a reverse alternate sequence 218 complementary to the forward alternate sequence. The two alleles (reference and alternate) for the particular SNP of interest have been determined by experiment and the relevant data is available from, for example, the U.C. Santa Cruz Human Genome Browser Gateway or the NCBI dbSNP website.

Each of the reference forward, reference reverse, alternate forward and alternate reverse sequences has a probe tiling associated with it, 220, 230, 240 and 250 respectively, represented as an array of squares in FIGS. 3A-3D, with each square representing a probe. Each tiling 220, 230, 240 and 250 includes twenty probes and so the tiling for the target SNP has a total of eighty probes. Although the probes are shown as being arranged contiguously and adjacently, it will be appreciated that in practice the probes can be dispersed provided that the position of each probe on the physical array is known. Each column of probes, e.g. column 222, corresponds to a probe set, comprising four probes, and each twenty probe tiling includes five probe sets. Each probe comprises a twenty five base long nucleotide sequence with the thirteen position as the interrogation position. Within each probe set, the interrogation position for each probe is substituted by a one of A, C, G or T as indicated in FIGS. 3A-3D. The first probe set, is used to interrogate the −2 position of the corresponding sequence, the second probe set the −1 position, the third probe set the SNP position (0), the fourth probe set the +1 position and the fifth probe set the +2 position. The probes for each tiling 220, 230, 240 and 250 are complementary, according to the base pairing rule, to the reference forward and reverse sequences and alternate forward and reverse sequences that they are respectively intended to interrogate.

For each probe set, the nucleotide at the interrogation position (position thirteen) is either A, C, G or T. The order is not relevant, provided it is know which probe is at which physical position within the probe set. The four probes of the first probe set are complementary to the target SNP sequence with the interrogation probe position interrogating the −2 position.

The probes tiling the array and their positions on the arrays are know and have been designed specifically for the reference allele sequences and alternate allele sequences at step 112 of the experimental protocol. As can be seen eighty probes in total are used for each target SNP. (Twenty probes each for the reference allele forward sequence, reference allele reverse sequence, alternate allele forward sequence and alternate allele reverse sequence.) However, an eighty probe tiling is presented herein by way of example and it will be appreciated by those of skill in the art that the invention is not limited to use of an 80 probe tiling and that more or fewer probes may be tiled for analysis of a target SNP. For example, one may choose not to interrogate the −2 or +2 positions and the resulting tiling might then contain only 48 probes (Twelve probes each for the reference allele forward sequence, reference allele reverse sequence, alternate allele forward sequence and alternate allele reverse sequence.)

In the following, ‘tiling’ will indicate, depending on the context, a number of probes used to interrogate a particular sequence. Thus, “tiling” may refer to the twenty probes for a reference allele forward 220, reference allele reverse 230, alternate allele forward 240 or alternate allele reverse 250 sequence individually, or to all eighty probes associated with the target SNP together. Alternatively, it may refer to a smaller number of probes used to characterize a target SNP.

Although a twenty five nucleotide probe sequence has been described above, it will be appreciated that other probe nucleotide lengths and probe formats can be used.

If conformance data is not required, as will be described further below, then a single probe, perfectly complementary to the respective reference forward, reference reverse, alternate forward and alternate reverse sequences, could be used for each tiling 220, 230, 240, 250 in which case four probes could be used. Fewer or more then the five probe sets per forward and reverse sequence of the reference and alternate alleles can be used, but five probes sets has been found to provide reliable conformance data as will be described below.

The conformance assessment process is carried out for all the non-rejected target SNP positions on all the arrays and for each individual used in the experiment. The following describes the conformance assessment process with reference to multiple eighty probe tilings, although extension of the general principle to other tilings will be apparent from the discussion presented herein.

A first of the multiplicity of eighty probe tilings is selected and the intensity data measured from the probes of the first tiling is used in the conformance assessment. The intensity data for the reference or alternate allele is selected and the intensity data for a first probe set, e.g. probe set 222 as indicated by the broken bold line, for the reference sequence is evaluated. The first probe set 222 interrogates the −2 position of the reference allele forward sequence 212. The location of the complementary probe which perfectly matches the reference sequence is looked up from stored data and the measured intensity or brightness for the perfect match probe location is determined from the intensity data. The perfectly matching probe 262 is at the third row of the first probe set. The perfect match probe intensity is then compared with the intensity data for the remaining probes in the probe set, to see if the measured intensity of any of the other probes in the same probe set is brighter, indicating that more DNA had bound thereto. In FIGS. 3A-3D, the emboldened probes indicate the perfect match probes in each probe set.

If the perfect match probe is determined to be the brightest probe in the probe set, then this is an indication that the probe has bound DNA having the intended sequence. Therefore that probe set can be considered to include useful experimental data and to be reliable. Therefore a count of the number of conforming probe sets for the reference tilings is incremented. The process then increments a count of the total number of probe sets for the tilings that have had their conformance evaluated. If a perfect match probe is not the brightest, then that does not necessarily mean that all the data for the tiling is unreliable, e.g. because it may be that the probes for that probe set are damaged or because there has been some other failure specific to that probe set, and the count of total number of probe sets evaluated is incremented to reflect that the probe set has been evaluated.

The process is then repeated for the next probe set 224 in the tiling 220 for the reference forward and reverse sequences and so on until all ten probe sets for the reference forward and reverse tiling have been evaluated. Then, the conformance for the reference sequence tilings 220,230 is calculated. In general, the conformance, C, for a tiling is the number of conforming probe sets divided by the total number of probe sets in the tiling, which in this example is ten.

Hence a conformance for the reference tiling, C_{R}, has been calculated. The process is then repeated using the intensity data for the alternate forward and reverse sequences and a conformance for the alternate sequence, C_{A}, is calculated. The conformance C for the target SNP is then set as the greater of C_{A} and C_{R} to reflect that although the data for the alternate or reference sequence may not be reliable, the data for the other may be reliable and so the conformance value for the better data is used as the metric by which to assess the validity of the data for the target SNP. The conformance value C is stored and indexed by the target SNP and an identifier of the individual. The conformance for a next target SNP is then calculated using the data for the corresponding tiling and the process is repeated until the intensity data for the 80, probe tilings for each SNP has been evaluated for an individual. Then the process may be repeated for the same or different SNPs from additional individuals.

In general, conformance measures the number of times that one of the reference or alternate sequence is indicated to be actually present out of the number of times either could have been present. It has been found that conformance values below about 0.8 tend to be an indicator that the tiling did not reliably detect the intended nucleotide sequence in the sample. A conformance threshold of 0.8 or greater can be used, but preferably a conformance threshold of 0.9 is used as a reliable indication that either the reference or alternate allele sequence was detected as present in the DNA sample.

The process is carried out for all of the non-rejected intensity data for all of the experiments to provide a data set of conformance values for each target SNP for each individual. The conformance values are thresholded and the data for those target SNPs having a conformance below the threshold conformance value, for example 0.9, are discarded or rejected as likely being unreliable data. This helps to improve the accuracy of the method by removing data from subsequent analysis which is believed not to correspond to a reliable detection of the intended DNA sequence. The use of a conformance metric to identify unreliable data is not necessary but can improve the accuracy of the method. The reduced intensity data set with the non-conforming data removed, or flagged as unreliable, is then used in the calculation of P′ values and genotyping of the individuals.

A process is then carried 208 out to determine a measure of the amount of each allele present at the locus, P′ (“p-hat”), for each SNP position using the conforming intensity data. The measure of allele amount, P′, is computed for each SNP position for each individual. In the case of a SNP being the genetic marker, and SNP positions generally being biallelic, either the reference allele for the SNP position of interest, or the alternate allele for the SNP position of interest will be present in a single strand of DNA from an individual. Thus, a diploid individual may possess two reference alleles, two alternate alleles, or one alternate and one reference allele. Either the reference allele or the alternate allele may be involved in a phenotype under investigation and it may be a combination of reference and alternate alleles at various SNP positions which results in the phenotype.

The actual relative allele amount, P, for a SNP position indicates the proportion of the reference and alternate alleles at the SNP position. For a diploid individual, at a biallelic locus, P should have one of three distinct values representing the homozygous alternate (all alternate allele), homozygous reference (all reference allele) and heterozygous (mixture of alternate and reference alleles) possibilities. However, it is not possible in practice to measure the actual allele amount precisely. In practice, the intensity of the emitted light from the probe sites is measured and is related to the amount of the particular alleles detected at that probe site, in a complex manner. Therefore an estimator, P′, of the relative allele amount is calculated from the measured experimental data and in general can be calculated using the intensity of light from the reference allele sequence (IR) probe as a proportion of the total intensity of light from the alternate and reference allele sequence probes (I_{A}+I_{R}), i.e. P′=I_{R}/(I_{A}+I_{R}), and so would be an estimated measure of the relative allele concentration defined by cᵣ/(cₐ+cᵣ). Optionally, the intensity measures can be corrected for background. The use of I_{R}/(I_{A}+I_{R}) rather than I_{A}/(I_{A}+I_{R}) is merely a matter of convention and the latter could be used in the alternate as an estimated measure of the relative allele concentration defined by cₐ/(cₐ+cᵣ).

The process of computing P′ values initially uses the intensity data for a first target SNP position from the target SNP positions whose data has not been rejected after the conformance test. In certain embodiments, P′ is calculated by averaging a set of P′ᵢ values, as described in greater detail supra. The data for the forward and reverse sequences can be processed separately, but in the illustrated embodiment the forward and reverse data are combined. The intensity data for the pair of perfectly matching probes from the first probe set for the reference and alternate sequences is identified. A perfect match probe pair is the set of two perfect match probes corresponding to a single offset (e.g. −2) and orientation (e.g. forward), one of which is from the reference tiling and the other of which is from the alternate tiling. The intensity measurements from these probes can be used to calculate a P′ᵢ value using the equation: I_{R}/(I_{A}+I_{R}). For example, as shown in FIGS. 3A and 3C, probes 262 and 264 comprise a perfect match probe pair. As such, a P′ᵢ value may be calculated using the intensities for the perfectly matching probe position 262 for the first probe set for the reference allele and the perfectly matching probe position 264 for the first probe set for the alternate allele. The perfectly matching probes for all the probe sets are represented by emboldened outlines in FIGS. 3A-3D.

A probe pair count is incremented and the process is repeated for each of the pairs of perfectly complementary probes in the tilings 220, 230, 240 and 250 for the current target SNP position. When all of the probe pairs have been evaluated, a P′ for the target SNP position is calculated as the sum of the P′ᵢ values (relative intensities) for each probe pair divided by the total number of probe pairs, which in this example is ten. This method of calculating a P′ for a target SNP position can be described as the “Mean of the Intensity Ratios” method. This P′ value indicates the relative amount of each allele present for that SNP in an individual. In further embodiments, rather than a standard (arithmetic) mean calculation, a trimmed mean may be used to calculate the P′ from the P′ᵢ values. A trimmed mean is found by ignoring the k smallest observations and the k largest observations and averaging the rest of the sample. In still further embodiments, a Winsorized mean may be used to calculate P′ from the P′ᵢ values. A Winsorized mean is similar to a trimmed mean except that instead of ignoring the k smallest and k largest observations, those observations are replaced by the nearest non-extreme values. For example, if k=2 the smallest and 2^{nd} smallest values are each replaced by the 3^{rd} smallest value, and the largest and 2^{nd} largest values are each replaced by the 3^{rd} largest value. The process is repeated for the next target SNP position until P′ values have been calculated for all the target SNP positions for all the individuals using the stored intensity data.

Alternatively, a P′ for the target SNP may be calculated by first averaging the intensities I_{R} and I_{A} for all the perfect match probes, and then calculating the ratio <I_{R}>/(<I_{A}>+<I_{R}>). This can be described as the “Ratio of the Mean Intensities” method. The average intensities may be calculated by a standard (arithmetic) mean calculation wherein all the perfect match intensities are summed, and then divided by the total number of perfect match intensity measurements for either the alternate or reference allele. In other embodiments, a trimmed mean or Winsorized mean calculation may be used. This method tends to reduce the sensitivity of the P′ calculation to outliers. As with the previously described method for calculation a P′ for the target SNP, the process is repeated for the next target SNP position until P′ values have been calculated for all the target SNP positions for all the individuals using the stored intensity data.

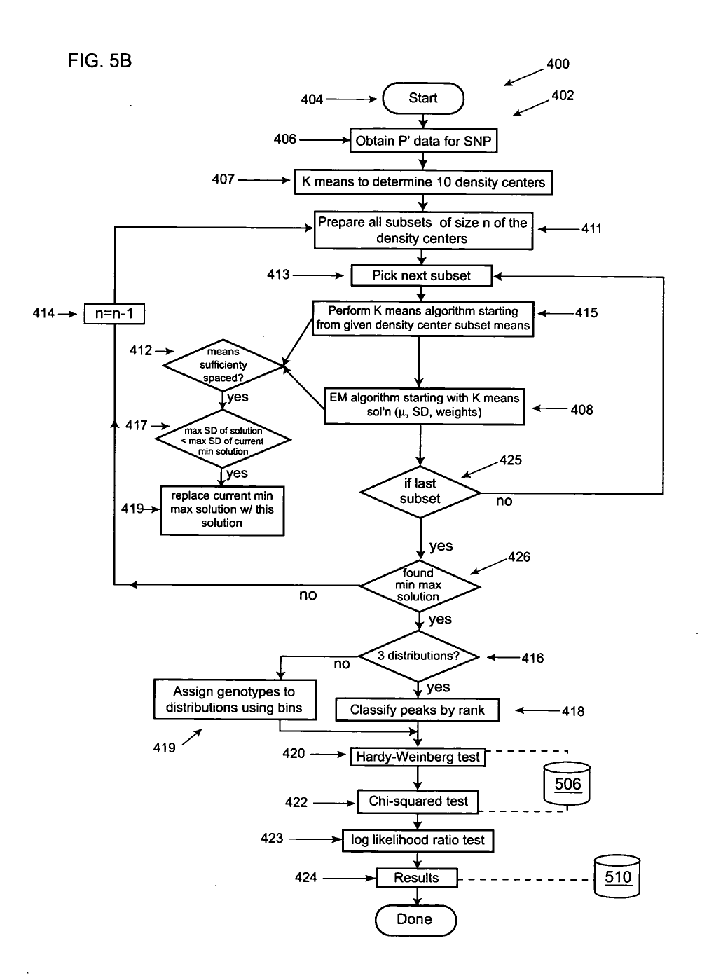

In another embodiment, 24 perfect match probes (comprising 12 perfectly matching probe pairs) may be used to calculate P′ values since there are four additional probes in the 80 probe tiling that are perfectly complementary to the reference or alternate sequence, considering that at position 0 (or offset 0) there is a perfect match to the reference sequence in the alternate tiling and a perfect match to the alternate sequence in the reference tiling. In other words, at the 0 offset for a particular tiling (reference forward, alternate forward, reference reverse, or alternate reverse), there are actually two perfect match probes, one that is complementary to the reference sequence and the other that is complementary to the alternate sequence. This is because at the 0 offset the tilings are duplicated between the reference and alternate tilings for a given strand orientation (forward or reverse). For example, in the reference forward tiling in FIG. 6 the top probe at the 0 offset is perfectly complementary to the reference sequence and the probe below the top probe is perfectly complementary to the alternate sequence. Therefore, the intensity measurements of the four additional perfect match probes (two additional perfect match probe pairs) may also be included in the P′ calculation for a given SNP, regardless of whether the “Mean of the Intensity Ratios” or the “Ratio of the Mean Intensities” method is used.

As one of skill will readily recognize, these methods may be combined in different ways. For example, one may use the “Mean of the Intensity Ratios” method with 12 perfect match probes, or one may use the “Ratio of the Mean Intensities” method with 10 perfect match probes. Further, either method may use trimmed, Winsorized or standard (arithmetic) means to calculate an average P′. In one embodiment, a background correction (as discussed supra) may be subtracted from the individual intensity measurements prior to calculating P′ values for a given SNP.

Therefore P′ values for each individual and for each SNP have been obtained and are ready for analysis in step 120 so that a genotype can be assigned to each individual for each of the SNP positions. When the individual genotypes have been assigned, general method 102 is done. The assigned genotypes can then be correlated with the phenotype under investigation or otherwise used in order to try and determine any relationship between the phenotypes of the individuals and their genomes.

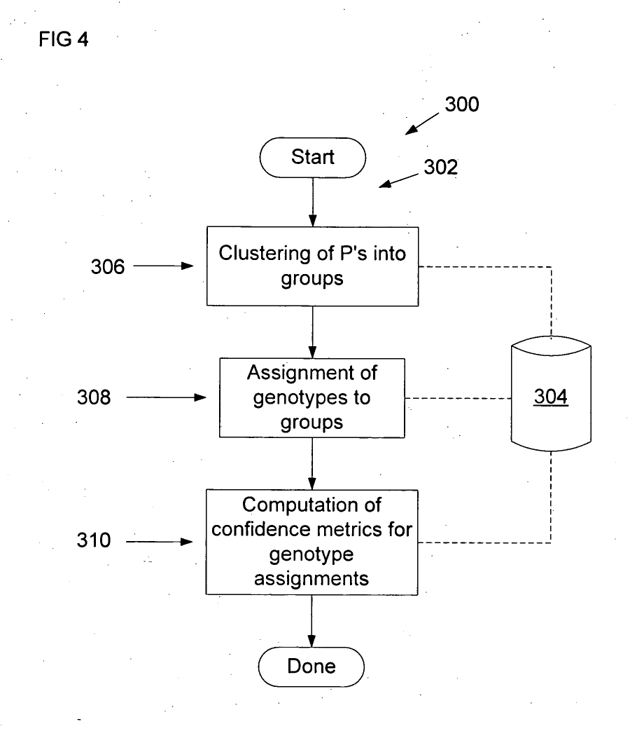

FIG. 4 shows a high level illustration of an embodiment of a software architecture 300 suitable for implementing a method according to the present invention, and corresponds generally to step 120 of method 102. A method of the present invention 302 is shown as three separate processes. However, this is merely for purposes of clarity of explanation and the invention is not intended to be limited to implementations comprising three separate processes only. Rather, the method of the present invention can be implemented in many ways in software, and the architecture shown in FIG. 4 mostly illustrates grouping of similar operations rather than being required. The method could be implemented as a single process or as more than three processes. Further, it will be apparent to a person of ordinary skill in the art from the following description, that various of the operations can be provided in the same or separate processes depending on the details of a particular embodiment of the general principles of the method as described herein.

The method 302 of the invention will be described in general at a high level with reference to FIG. 4 before a more detailed description of aspects of the method will be presented. As illustrated in FIG. 4 the architecture 300 generally comprises the computer implemented method 302 and a database 304 of data items used by the various processes of the method. The database 304 can be a relational database. An example database schema for an embodiment of the database is described below with reference to FIG. 6. In one embodiment, the database is a relational database, e.g., as provided by Oracle Corporation of California. The database 304 includes a database management system (DBMS). Interactions between the processes of the method 302 and the DBMS are represented in the figures by dashed lines, although not all the database interactions will actually be shown in the figures for the sake of clarity.

The method 302 can comprise three main processes or modules. A first module or process 306 clusters P′ values into a number of groups using a probability clustering process. In an embodiment, the clustering process 306 is written in an object-oriented programming language, such as Java (Java is a trade mark of Sun Microsystems Inc. and is registered in some countries) or J# (J# is a trade mark of Microsoft Corporation and is registered in some countries). A second module or process 308 assigns a particular genotype to each of the groups resulting from the clustering process. In an embodiment, the genotype assigning process 308 is carried out using a SQL update query to the database 304. The result of the genotype assigning process 308 is that a genotype has been assigned to the individuals for a genetic marker or markers. A third module or process 310 can optionally be provided to determine the confidence in the assignments of the genotypes to the individuals for the genetic marker or markers. In an embodiment, the confidence determination process 310 includes calculating at least one confidence metric which can be used to reject, or filter out, genotype assignments which are considered unreliable. In an embodiment the confidence determining process 301 is written in an object-oriented programming language, such as Java or J#. The result of the confidence determination process is the assignment of a genotype to an individual for the genetic marker or makers that is considered reliable to a particular confidence level.

For relatively small groups of individuals, e.g., eighteen individuals, the method has been found to provide incorrect assignments of an individual's genotype at a level of approximately 1%. For larger groups of individuals, e.g., two hundred and thirty five individuals, the method has been found to provide incorrect assignments of genotypes to individuals at a level approaching zero percent. Hence the results generated by the method can be considered to be highly reliable.

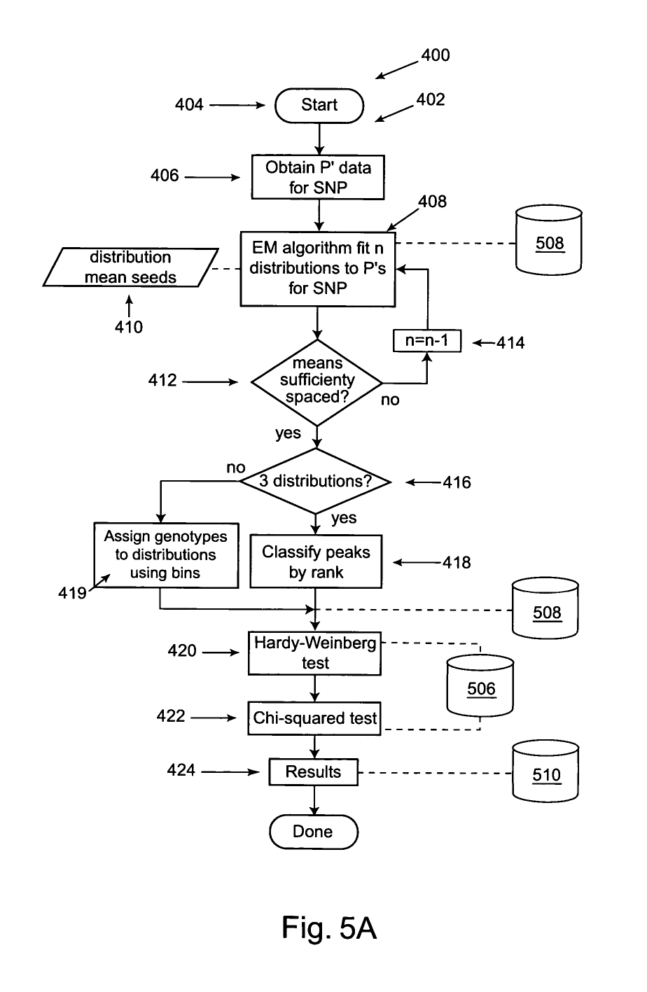

The general method 302 and processes will now be described in greater detail with reference to FIGS. 5 and 6. The flow chart 400 shown in FIG. 5 illustrates various operations carried out by processes 306, 308 and 310. However, in FIG. 5, flowchart 400 further illustrates the process control flow of a single process 402 and combinations of the various operations carried out by process 402 correspond to the separate processes 306, 308 and 310. FIG. 5A illustrates an embodiment employing a probability clustering process, e.g., an expectation maximization process. FIG. 5B illustrates an embodiment employing both probability clustering and distance-based clustering (e.g., K-means) processes. FIG. 6 shows an embodiment of a schema 500 of database 304. An embodiment of the database uses five tables.

A first table 502, EM_Genotyping analysis, is used for data items relating to a particular genotyping analysis carried out by the process 402. This table defines a particular genotyping analysis. The table includes fields storing data items representing a number of properties and attributes of a particular genotyping analysis carried out by the process 402. A Genotyping_analysis_id data item represents a unique identifier for a particular genotyping analysis. A analysis_name data item represents a name for the particular genotyping analysis. A genotyping_analysis_type data item represents the type of genotyping analysis carried out by the process. A project_name data item represents an identifier for a project with which a particular genotyping analysis is associated. A created_dt data item represents the date and time on which the analysis item is created and a analyzed_dt data item represents the date and time on which an analysis is finished.

A second table 504, EM_Genotyping_analysis_scan, is used for data items indicating the experimentally derived data that is passed to the process for analysis. This table contains a list of scan_experiment_id which represents a unique identifier for the experimental scan data for each gentotyping_analysis_id data item. The scan_experiment_id data item is passed to the process 402 and the process uses it to obtain the appropriate experimental data required by the process from a different part of database 304, or from a different secondary storage location or device.

Tables 502 and 504 between them define the genotyping analysis that is to be carried out. When the process 402 is run, genotyping_analysis_id is provided to the program as an argument and process 402 expects tables 502 and 504 to be populated for the genotyping_analysis_id.

The process 402 writes the genotyping results data to three other tables 506, 508 and 510. The third table 506, EM_Genotype_snp_data, has fields for storing data items relating to the genetic marker, which in the described embodiment is a SNP position. As well as the genotyping_analysis_id data item, table 506 includes a snp_id data item which represents the particular snp position with which the data items are associated. A num_scans data item indicates the number of experimental scans and therefore corresponds to the number of individuals. A num_clusters data item represents the number of clusters finally identified by the process. A hardy_weinberg_p_value data item represents the p-value, or confidence, resulting from a genotype assignment confidence test as will be described in greater detail below. A min_chi2_p_value_{—}0_{—}05 data item represents the p-value resulting from a further genotype assignment confidence test as will also be described in greater detail below. A final_log_likelihood data item represents a final likelihood of the distribution assignments or fits from the probability clustering.

The fourth table 508, EM_Genotype_cluster_data, has fields for a number of data items relating to the clusters to which P′ values are assigned. This includes the genotyping_analysis and snp_id data items as well as a cluster_number data item which identifies a particular cluster. A mean data item, a standard_deviation data item and a weight data item respectively represent the mean, standard deviation and weight of a particular cluster of P′ values and are derived from the probability clustering process as will be described in greater detail below. An assigned_genotype data item represents the genotype assigned to a particular cluster. A chi2_p_value_{—}0_{—}05 data item represents the p-value for a particular cluster corresponding to a chi-squared values calculated from the standard deviation for the cluster.

The fifth table 510, EM_snp_genotype, has fields for data items relating to the results of the process of probability clustering and in particular, each P′ is assigned a cluster number. The table includes a number of the above mentioned data items, a snp_design_block_id data item uniquely the SNP tiling on the SNP chip (e.g., one SNP can be tiled more than once on a chip and thus, the SNP_id is not sufficient information to distinguish the SNPs); and a cluster_log_likelihood data item representing log likelihood of the individual P′ value in the assigned cluster.

In order to run process 402, a command line instruction to execute the program is entered together with arguments, genotype_analysis_id and any cut-off for the distance between means of neighboring clusters or distributions. It should be noted that these cut-off may vary with marker. After the process has initiated 404, the process obtains P′ data for a SNP of interest 406 for all the individuals from the database using scan_experiment_id to locate the P′ data for the SNP of interest. The P′ values are then made available to a one or more of a probability clustering process and a distance-based clustering process. As shown in FIG. 5A, a probability clustering process 408 attempts to assign the P′ values to a number of clusters or groups. In an embodiment the probability clustering process is an expectation maximization algorithm (hereinafter EM algorithm).

The EM algorithm tries to assign the P′ values to a number of clusters by fitting a mixture of normal distribution functions to the P′ values. Each distribution function is characterized by its mean, its standard deviation and its weight. The weight of a distribution corresponds to the probability of finding a one of the P′ values in the distribution and corresponds to the area under each distribution. The weight of a distribution indicates the proportion of the total number of P′ values present in that distribution. The sum of the relative weights is unity, reflecting the fact that all of the P′ values must fall in the resulting, fitted distributions. Further details as to the properties and operation of the EM algorithm are available in DATA MINING: PRACTICAL MACHINE LEARNING TOOLS AND TECHNIQUES WITH JAVA IMPLEMENTATIONS, by Ian H. Witten and Eibe Frank, pub. Morgan Kaufmann (1999), ISBN 1-55860-552-5, which is incorporated herein by reference in its entirety.

Initially the EM algorithm attempts to assign the P′ values for all the individuals into three groups by fitting three distributions to the P′ values, one for each of the potential genotypes possible: homozygous reference (r); homozygous alternate (a); and heterozygous (h). The EM algorithm is an iterative process. Conventionally it starts by randomly assigning members of the data set being fit to a one of the distributions, determining the corresponding distribution parameters and then re-assigning the members of the data set. However, this approach can result in the algorithm becoming trapped in a local minimum solution when in fact there is a better, global minimum solution. It has been found that it can be beneficial to supply seed values to the EM algorithm in order to help prevent it becoming stuck in a local minimum. Therefore, in an embodiment, seed values 410 are supplied to the EM algorithm 408 which are used in the first fit to the P′ values.

In particular, the seed values comprise a mean P′ value for each of the distributions being fit. As will be described further below, the process 402 initially fits three distributions. However, if it is determined that three clusters or groups of P′ values appear an unreliable clustering, then the process reapplies the EM algorithm but using only two distributions. Similarly, if two clusters appears unreliable clustering, then the process 402 applies the EM algorithm using a single cluster. Therefore, the number of distribution mean seeds 410 supplied depends on the stage in the processing. In a first instance, three distribution mean seeds are supplied. In one embodiment the three distribution mean seeds are approximately 0.2, 0.5 and 0.8, with the ratio between the three distribution mean seeds being 1:1:1. In a second instance, two distribution mean seeds are supplied. In one embodiment the two distribution mean seeds are approximately 0.3 and 0.7, with the ratio between the two distribution mean seeds being 1:1. In a third instance, one distribution mean seed is supplied. In one embodiment the one distribution mean seed is approximately 0.5.

The EM algorithm initially fits three distributions to the P′ values, using the three distribution mean seeds, and returns three means {μ1, μ2, μ3}, three standard deviations {σ1, σ2, σ3} and three weights {p1, p2, p3; p1+p2+p3=1} and the corresponding data items are kept in memory with the final results being written to database 510.

In alternative embodiment (illustrated schematically in FIG. 5B), rather than providing seed values to prevent the EM algorithm from becoming fixed in a suboptimal local minimum, a distance-based or probability clustering process is utilized. In a favorable embodiment, a K-means algorithm is employed. K-means clustering is classified herein as a distance-based clustering process, in contradistinction to probability clustering processes exemplified by the EM algorithm. K-means clustering assumes that the data is distributed into non-overlapping clusters, and that each member of a data set is assigned to a single cluster. The K-means algorithm then finds locally optimal solutions minimizing the sum of the distance squared between each data point and its nearest cluster center. Like the EM process, K-means is an iterative refinement process, which seeks through successive repetitions to determine the distribution of members of a data set into distinct classes.

In the methods of the invention, the K-means process begins with assigning values to a plurality of means, or density centers. Most commonly, values (between 0 and 1, as described above with respect to the EM clustering algorithm) are assigned for 10 evenly distributed density centers. Although it would be equally possible to assign values to any finite number of density centers greater than 3 (e.g., between 3 and 1000), it will be obvious to one of skill in the art that the optimal number, while arbitrary, provides adequate discrimination while not requiring unnecessarily extensive computing resources. Following assignment of mean values, the K-means algorithm 407 iteratively distributes the data between 10 means to establish 10 density centers.

The process then prepares all possible subsets of 3 (or 2 or 1) density centers 411 obtained by the K-means algorithm 407 and uses them for the seed values for the probability, distance-based or both clustering. In this manner, in a preferred embodiment; the K-means process determines the distribution of data into clusters for all possible combinations of subsets of the 10 density centers identified by the first K-means process 407. The solutions, defined as the means, standard deviations and weight values determined following two consecutive cycles of calculation and assignment with no data points altering membership in their assignment to a cluster, then provide the initialization points for performing the EM 408 (or other probability clustering) processes described above. It should be noted that each EM process is typically initiated using the solution obtained for each combination of density center subsets. While performing sequential K-means algorithms using 1) 10 assigned means; and, 2) subsets of n density centers obtained by the first K-means process, offers a significant improvement over prior methods, one of skill in the art will recognize that a single K-means step (i.e., using 10 assigned means, or subsets of each combination of n assigned means) will offer some benefit relative to initializing the EM algorithm using seed values. This process is repeated 425 for each subset of density centers until the last subset 425 has been completed.

As in the methods described above employing seed values, the method is initially applied to subsets of 3 density centers. A distribution corresponding to 3 density centers is deemed reliable 412 for a given data set, if the means for each cluster are separated by greater than 0.1241 (mean P′ values greater than 0.1241 apart). In the event that subsets of 3 density centers yield an unreliable clustering, subsets of 2 and 1 density center are sequentially utilized 414, and evaluated against the same criterion.

For each set of means corresponding to the K-means solution for each combination of density center values, the standard deviation for each distribution is calculated and maintained in memory for later reference. Again, following execution of the EM algorithm, a standard deviation for each distribution is similarly calculated and maintained in memory. In addition, the maximum standard deviation for each of the 3 data clusters is computed and evaluated.

The maximum standard deviation for each subset is then compared with the maximum standard deviation obtained for the prior subset in a pair-wise comparison 417 to determine the K-means or EM solution providing the minimum maximum standard deviation. If the subsequent maximum standard deviation is less than the current maximum standard deviation, the former value is rejected in favor of the subsequent value 419. Alternatively, all values can be recorded and compared at the completion of the K-means algorithm for each subset of density centers; however this method is computationally more complex and memory intensive than the above described consecutive pair-wise comparison. Similarly, it is possible to employ the minimum average standard deviation, although this tends to be a somewhat less rigorous approach because narrow standard deviations within a subset tend to compensate for broader standard deviations within the same subset. For each subset of density clusters the standard deviations for the K-means and EM solutions are likewise compared, and the solution yielding the minimum maximum standard deviation is selected 419 as the preferred solution for that subset of density centers. Upon completion of all the subset combinations, and ascertainment of the minimum maximum standard deviation 426, the process proceeds to step 416 as described below.

As described above, each of the clustering processes, whether probability or distance-based, is initially performed assuming that the data is distributed into three clusters. Regardless of the embodiment, the process 402 determines 412 whether the identification of three clusters is reliable or not. A conventional cross validation method has been found not to work reliably for the application of the EM and K-means algorithms to individual genotyping. Instead a method based on cut-off values is used by the process 402. The process determines whether the values of the means for the distributions are sufficiently separated in order for the assignment of three clusters to the P′ values to be considered valid. The process calculates the differences in the means of adjacent distributions, i.e. Δμ12=|μ1-μ2| and Δμ23=|μ2-μ3|, and determines whether either of Δμ12 or Δμ23 is less than a threshold value. If so then the clusters are considered to be too close to be reliably identified as separate clusters, indicating that the P′ values assigned to different clusters should more likely have been assigned to a single cluster. An example method by which the threshold value can be determined is described below. In one embodiment a threshold value of approximately 0.1 can be used. This threshold value has been found to be particularly suitable for analyzing P′ values derived from intensity measurements with fluorescein as the detectable marker. However, it has been found that the distribution (spread) of P′ values can vary depending on the detectable marker used in the experiment from which they have been derived, and as such, the distance between the means of adjacent distributions may be larger or smaller depending on the detectable marker utilized. Therefore, the marker scaling argument can be used to scale the threshold value to take into account variations in the distribution of P′ values with the marker used in the experiments. For example, the range of P′ values with cychrome as the marker was found to be larger than when fluorescein was used as the marker. Based on this, the scaling factor was empirically determined to be 1.2407 between fluorescein and cychrome. Therefore a difference between mean threshold of 0.1241 is passed to the process 402 when it is initiated.

If the separations of the means of adjacent clusters are found to exceed the threshold, then process flow proceeds to step 416. Alternatively, process flow branches, the number of clusters to be fitted, Nfit, is reduced by one 414 and the clustering algorithm is carried out again 408, but fitting only two clusters or groups and using two distribution mean seeds 410. The difference in the means of the two distributions is again compared with the threshold 412 to determine if the distributions are sufficiently separated. If not, then process flow again branches and the clustering algorithm is carried out 408 fitting to only one cluster and using one distribution mean seed value 410. Steps 404 to 412 are equivalent to module 306 of FIG. 3.

The process then assigns genotypes to the clusters identified. The process determines 416 whether three distributions, corresponding to assigning the P′ values to three clusters, were identified by the clustering algorithm. If the P′ values were assigned to three clusters, then a first genotype assignment process is used. If less than three clusters were assigned then a second genotype assignment process is used. If three clusters were identified, then the genotype can be assigned based on the order or rank of the clusters. The clusters are ordered by their respective means and the first ranked cluster (lowest mean) is assigned the genotype of homozygous alternate, the second ranked cluster (middle mean) is assigned the genotype of heterozygous and the third ranked cluster (highest mean) is assigned the genotype homozygous reference. It will be appreciated that the order of assignment of genotype will be reversed if P′ is calculate as the quotient of Ia and Ia+Ir.

If it is determined 416 that less than three clusters were identified, then process flow branches and genotypes are assigned 418 based on the range of P′ values in which the distribution means of each distribution fall. In the described embodiment, P′ can vary between approximately 0 and 1 (i.e., the range extends slightly beyond 0 and slightly beyond 1, while remaining within the value expected for noise). A first range of values (0 to 0.32) or bin corresponds to a homozygous alternate genotype, a second range of values (0.32 to 0.68) or bin corresponds to a heterozygous genotype and a third range (0.68 to 1) or bin corresponds to a homozygous reference genotype. These range values have been found to be appropriate for data derived from a fluorescein marker. The ranges of 0 to 0.38, 0.38 to 0.62 and 0.62 to 1 have been found to be appropriate for data derived from a cychrome marker. An example of a method for calibrating the range of P′ values to allow genotype assignments in this manner is described below. In some instances, two clusters may fall into the same genotype P′ range. When this occurs, if the range in which two clusters fall is the homozygous alternate range, the cluster with the larger mean P′ will be genotyped as heterozygote. Alternatively, if the range in which two clusters fall is the homozygous reference range, the cluster with the smaller mean P′ will be genotyped as heterozygote. Finally, if the range in which two clusters fall is the heterozygous range, the cluster with the mean P′ closest to 0.5 will keep the heterozygote genotype and the other cluster will be assigned a homozygous reference genotype if its mean P′ is greater than 0.5, or will be assigned a homozygous alternate genotype if its mean P′ is less than 0.5. Hence a genotype has now been assigned to each of the clusters of P′ values and therefore the genotype for each individual for this SNP has now been determined. The appropriate data items can be written to table 508 of the database 304. Steps 416 to 418 correspond generally to module 308 of FIG. 3. It should be noted that these are global cutoffs general to all SNPs, but alternatively, one can derive cut-offs on a per SNP basis.

It will be appreciated that in the instance of assigning a genotype to a single individual, only a single cluster will exist and so the genotype is assigned based on the genotype bin into which the P′ value for the individual falls and only a single cluster is fitted. Similarly when there are only two individuals, initially two clusters are fitted and the clusters or cluster to which the P's are assigned are assigned a genotype based on the bin or bins in which the cluster mean, or cluster means, fall.

In order to improve the reliability of the assignment of genotypes to the individuals, a test or tests can optionally be carried out to determine the confidence in the genotype assignment. In one embodiment, a confidence test based on Hardy-Weinberg equilibrium 420 and a confidence test based on a chi-squared distribution 422 together with a next_max_log_likelihood_ratio are carried out in order to reject any unreliable genotype assignments. In one embodiment, a genotype assignment is only considered a reliable result if it passes both of the confidence tests and has relatively large next_max_log_likelihood_ratio. The next_max_log_likelihood_ratio is defined as the ratio between the likelihood of a measure of the amount of an allele corresponding to the assigned group and the likelihood of the measure of the amount of an allele corresponding to the next best fit group. In other embodiments passing either test may be sufficient and more or fewer that two tests, using different statistical analysis methods, can be used. After both confidence tests have been applied for each of the SNPs, the appropriate result data items summarizing the genotyping analysis are written 424 to table 510 of the database. Steps 420, 422 and 424 correspond generally to module 310 of FIG. 4.

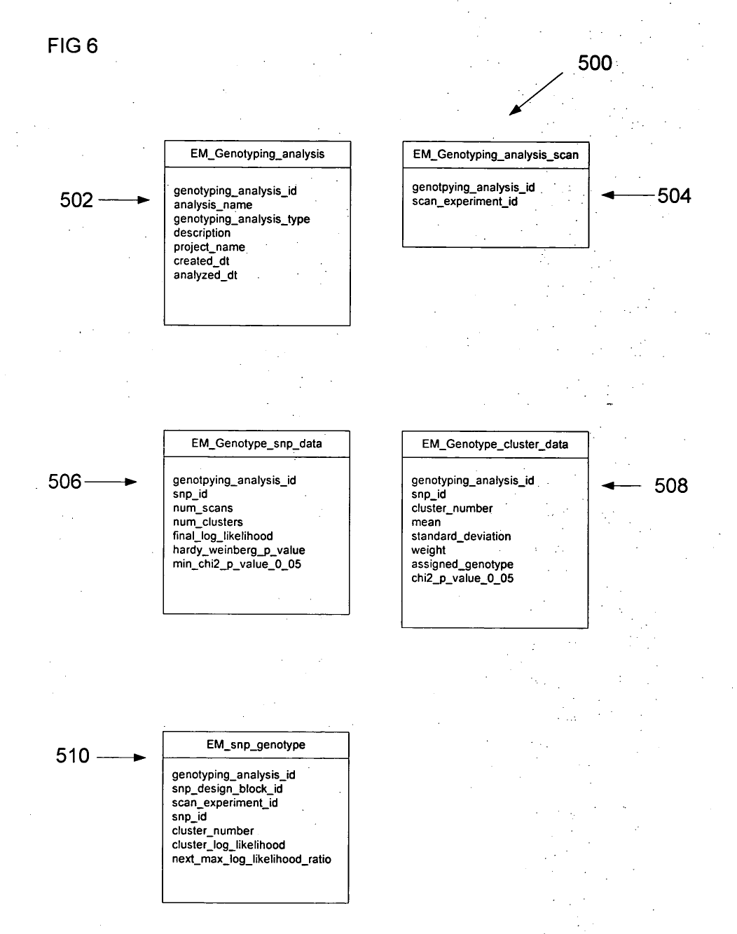

A process for carrying out the first genotype assignment confidence test 420 based on the closeness of the clustering of the P′ values to an expected Hardy-Weinberg equilibrium distribution for the same number of individuals will now be described in greater detail with reference to FIG. 7. FIG. 7 shows a flow chart 600 illustrating a process 602 for carrying out a first genotype assignment confidence test and corresponds to step 420 of FIG. 5. In general terms, the test is based on determining whether the assignment of the P′ values for the individuals to clusters can be considered to be consistent with the expected Hardy-Weinberg equilibrium distribution or frequencies of genotypes for the same number of individuals.

Hardy-Weinberg equilibrium, in general, is understood by persons of ordinary skill in the art. It relates the relative allele frequency to genotype frequency in a population. If there are two alleles for a locus, in this example the SNP reference and alternate alleles, with frequencies p and q respectively, then the predicted frequency of homozygous reference is p², or homozygous alternate is q² and of heterozygous is 2pq. For a diploid organism, a population of N individuals will be produced from 2N gametes and contain 2N alleles for each genetic locus, in this example a SNP position. The number of reference alleles (nᵣ) is twice the number of homozygous reference (nₕᵣ) plus the number of heterozygous (nₕₑₜ). The frequency of the reference allele is the number of reference alleles divided by 2N, and similarly for the alternate allele. Therefore, p=(2nₕᵣ+nₕₑₜ)/2N, q=(2nₕₐ+nₕₑₜ)/2N, nᵣ+nₐ=2N and p+q=1. If there are more than two alleles of a genetic marker, then they can all be counted in a similar manner.

Returning to process 602, a first SNP position is selected for evaluation 604. The process then determines 606 every possible way or permutation for distributing the number of individuals, N, amongst three clusters, including all individuals in a single cluster. The process does not need to repeat the determination for symmetric cases. The process then calculates a Bayesian factor (Bf) 608 for every one of the permutations identified in step 606. The calculation of Bayesian factors is described in “An Unconditional Exact Test for the Hardy-Weinberg Equilibrium Law: Sample-Space Ordering Using the Bayes Factor” by Luis E. Montoya-Delgado et al, Genetics 158: 875-883 (June 2001), and which is incorporated herein by reference in its entirety for all purposes. The calculated Bayesian factors are then sorted 610 by size and the sum of all the Bayesian factors, Σ_{Bf}, is calculated 612. The process 602 then determines 614 the Bayesian factor (Bf*) for the actual distribution of the individuals assigned amongst the clusters for the SNP, using the number of individuals assigned the various genotypes, i.e., N_{ref}, Nₐₗₜ, and Nₕₒₘ 616. The process 602 the calculates a second Bayesian factor sum 618, Σ_{Bf*}, using the Bayesian factors calculated previously, starting from the smallest Bayesian factor and up to and including Bf*.

A p-value, or confidence level, is then calculated 620 and is equal to Σ_{Bf*}/Σ_{Bf}. A high p-value is indicative of a high confidence that the actual assignment of genotypes to the individuals in the clusters corresponds to a distribution predicted by Hardy-Weinberg equilibrium and so the assigned genotypes can be considered reliable. The process then thresholds the calculated p-value to determine 622 whether the confidence level is sufficient for the genotype assignments for the SNP to be considered reliable. In one embodiment a 99.9% confidence level is used and a p-value threshold of 0.001 is used so that if the p-value is less than 0.001, the genotype assignments for the SNP are rejected 624. Otherwise the genotype assignments are accepted. The process 602 can then be repeated 628 for any SNP positions which have not yet been evaluated. Therefore this first test assesses whether the weights of the clusters conforms to that predicted by the Hardy-Weinberg equation and so helps to identify bad clustering of P′ values.

It is possible that by chance the clusters of P′ values would meet the Hardy-Weinberg equilibrium based test and so a further confidence test 422 can be used to help identify incorrectly assigned genotypes. This test evaluates the spread of P′ values in a cluster to help determine whether the cluster assignment was incorrect. For example, only two clusters may erroneously be assigned by the EM algorithm to a set of P′ values, which on visual inspection, can be seen to have three discernable clusters. Therefore the spread of P′ values in a one of or both the assigned clusters can be indicative of this incorrect cluster assignment. This test uses a chi-squared distribution to decide whether the spread of each cluster is greater than an expected cluster spread, and takes into account the number of individuals assigned to the cluster. The number of P′ values in a cluster determines how much the cluster spread can vary from an expected cluster spread, before the cluster can be considered unlikely to be reliable.

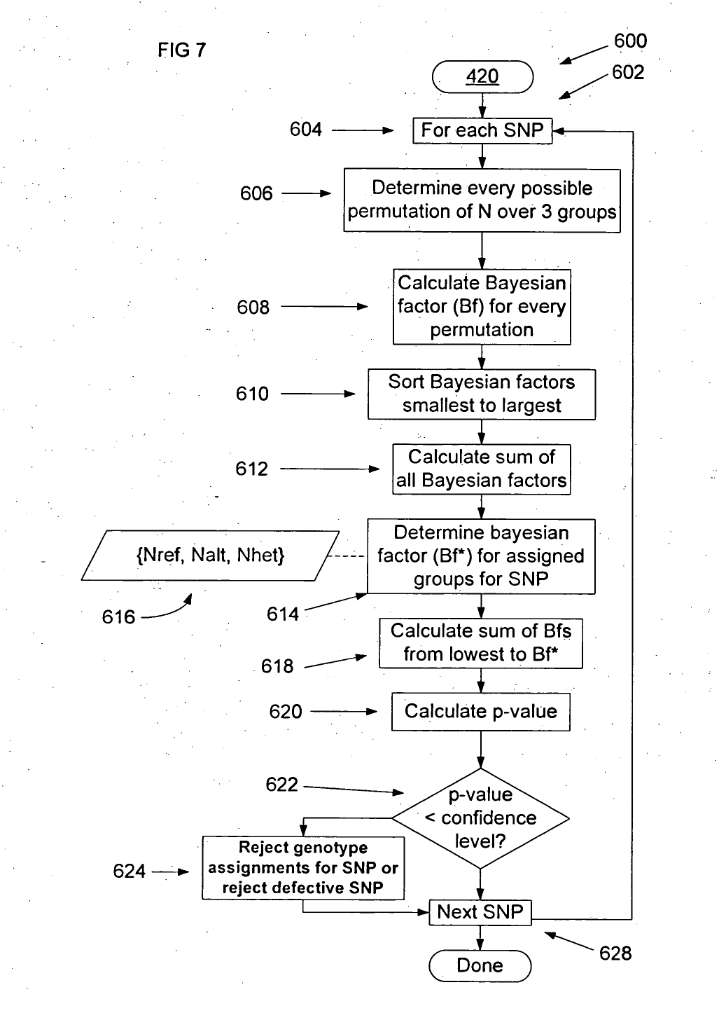

FIG. 8 shows a flow chart 700 illustrating a process 702 for carrying out a second genotype assignment confidence test and corresponds to step 422 of FIG. 5. The process 702 selects a first SNP position for evaluation 704. The process then selects a one of the genotypes 706 that has been determined to exist for the SNP (one, two or three genotypes can be identified for each SNP) for evaluation. The number of degrees of freedom (DofF) for the genotype is then calculated 708 and corresponds to the number of P′ values assigned to the cluster corresponding to the genotype (N_{ref}, Nₐₗₜ or Nₕₒₘ) minus 1. A chi-squared value is then calculated for the cluster using the expression χ²=(σ²×DofF)/(0.05)², where σ² is the variance for the cluster and 0.05 is the largest acceptable standard deviation for any cluster. Other largest acceptable standard deviations can be used depending on the details of the data being analyzed and the experiments from which the P′ values originated. Using the chi-squared value, a p-value for the number of degrees of freedom is determined 712, by calculation or using a look up table. A standard deviation of 0.05 has been found empirically to be a reasonable maximum standard deviation for a genuine genotype cluster of P′ values. Therefore the null hypothesis being tested is that the standard deviation of the actual sample cluster can be considered to be the same as the largest acceptable standard deviation of the underlying population cluster.

At step 714 the confidence in the spread of the cluster being that of a genuine cluster is determined by comparing the p-value with a threshold value. In an embodiment a threshold value of 0.001 is used. Other p-value thresholds can be used depending on the confidence level required. If the p-value is less than 0.001 then this corresponds to a 99.9% confidence level that the standard deviation of the cluster is too great to be the standard deviation of a genuine cluster and so the assignment of the P′ values to that single cluster is determined to be unreliable 714. Alternatively a p-value greater than 0.001 can be considered an indication that there is sufficient confidence in the standard deviation of the cluster corresponding to the standard deviation of a genuine cluster that the assignment of the P′ values to that single cluster can be considered reliable. If any one of the clusters for a SNP position is determined to be unreliable, then the genotype assignments for the SNP as a whole can be determined to be unreliable.

The process then determines 718 whether there are any assigned genotypes and associated clusters that remain to be evaluated for the current SNP position, and if so steps 708 to 718 are repeated. After all the clusters for a current SNP have been evaluated, the process 702 determines whether there are any SNP positions remaining to be evaluated 720 and if so steps 706 to 718 can be repeated. Otherwise the process 702 ends.

Various data items generated by the Hardy-Weinberg and chi-squared test are written to tables 506 and 508 of the database during processes 602 and 702. In process 424, the process 402 marshals relevant data items and writes them to table 506 or 508. It should be noted that table 510 is populated by the EM probability clusters and that there are no genotype assignments or statistical test results in table 510. Rather Table 510 contains the cluster number for all SNPs. More or fewer confidence tests, and of different type to those described above, can be used in other embodiments. Also different confidence levels can be used.

An embodiment of a process by which a cluster mean cut-off value for use in the above described process for determining the number of clusters to which to assign P′ values will now be described with reference to figures F and G. This process is separate to the above described individual genotype analysis process 402. In general terms, the process applies the probability cluster process to two independently measured data sets for the same individuals and minimizes the number of inconsistently assigned genotypes as a function of a cluster or distribution mean cut off.

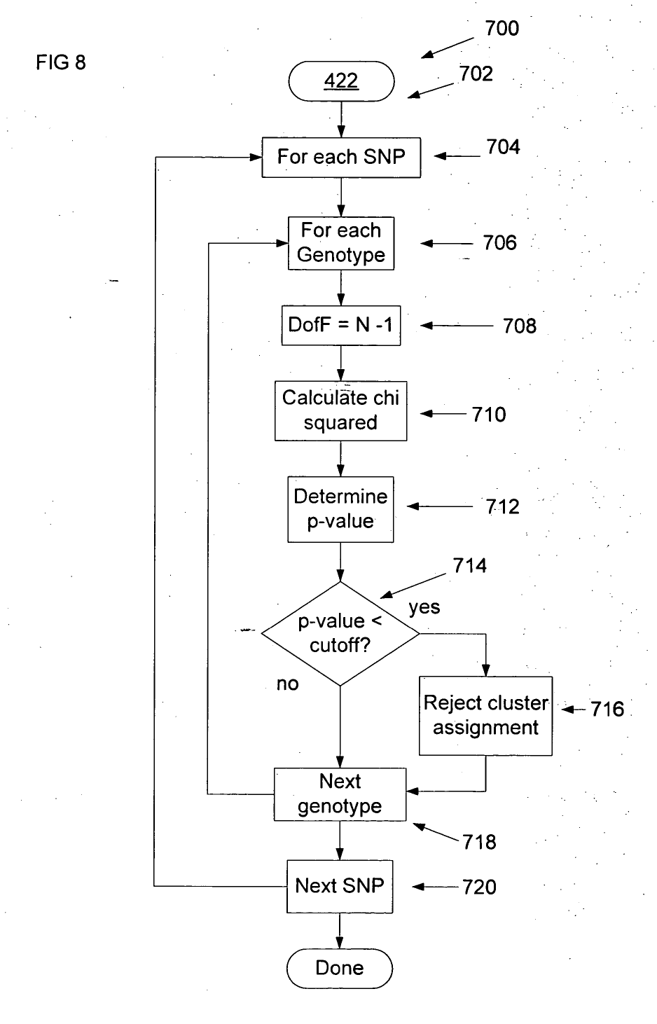

FIG. 9 shows a flow chart 800 illustrating a process 802 for determining a distribution mean separation cut off for use in determining the number of clusters to which to assign a set of P′ values for a group of individuals. The process initially sets a cut off 804 at an initial value. In one embodiment the cut off can initially be set at approximately 0.1. The process 802 then acquires a first set of P′ values 806 which were measured for a relatively large group of individuals, e.g. a few tens or hundreds of individuals. The EM algorithm is applied 808 to the first set of P′ values and initially tries to assign the P′ values to three clusters. If any of the separations of the mean values of the clusters assigned is less than the current cut off, then the EM algorithm is applied again to assign the P′ values to two clusters and then only one cluster if the two clusters are separated by less than the current cut off. This process 808 is similar to steps 408 to 414 of process 402. The first set of P′ values can include P′ values for a single or multiple SNP positions. Process 808 can then assign clusters for a single SNP or each of the SNP positions depending on the P′ data available.

The process 802 then acquires a second set of P′ values 810 for the same group of individuals as the first set 806, but obtained from different experimental data. For example, the two sets of P′ data can be obtained from separate experiments and separate scans of probe arrays. Alternatively, the two sets of P′ data can be obtained from scans of the same physical probe array but by acquiring intensity data at different wavelengths from two different markers, e.g. fluorescein and cychrome. Irrespective of how the two independent P′ data sets for the same individuals are obtained, the process then assigns the second set of P′ values to clusters using the EM algorithm and current cut off in a process 812 analogous to process 808.

The process 802, then assigns first genotypes 814 to the clusters identified for the first set of P′ values using a process analogous to steps 416 to 419. Genotypes are assigned for each SNP position. Second genotypes are then assigned 816 for the clusters identified for the second set of P′ values using a process analogous to process 814 as described above, and for each SNP position. The process then determines 818 whether the genotypes assigned are consistent. As the P′ values are obtained from data for the same physical individuals, the genotypes of each individual is the same. Therefore discrepancies in the assigned genotype should be owing to an erroneous genotype assignment by the process.

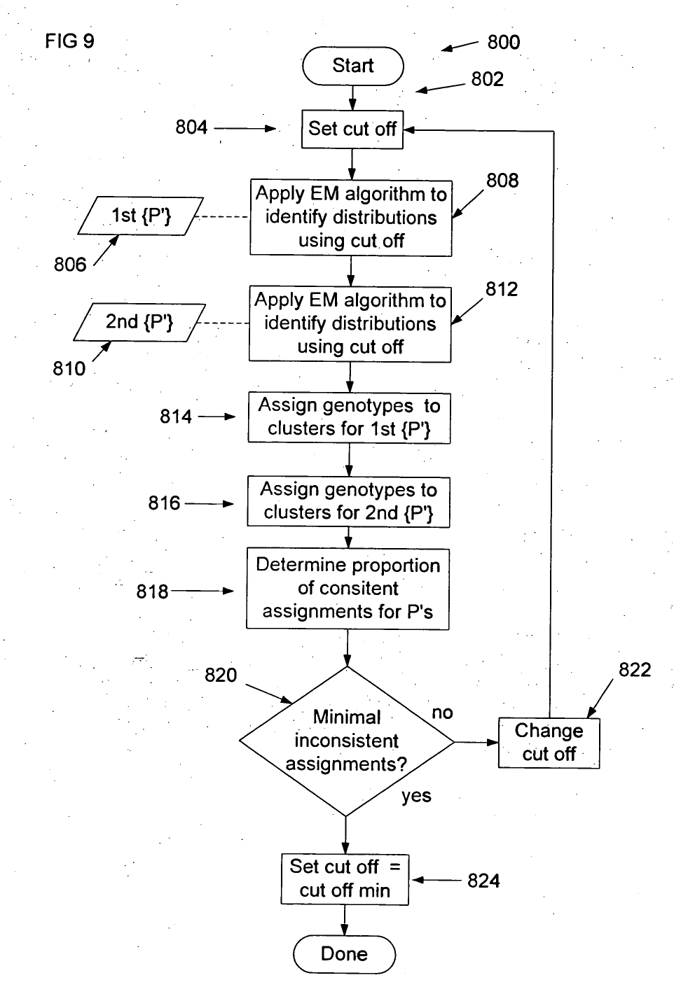

FIG. 10 shows a flow chart 850 illustrating a process 852 for carrying out step 818 of process 802 in greater detail. A SNP position is selected for evaluation 854 and a particular one of the individuals is also selected. The process 852 then determines whether the genotype assigned to that person at step 814 using the first P′ value is the same as the genotype assigned to the same person at step 816 using the second P′ value. If the genotypes are the same then the genotype assignment is consistent and a count of consistent genotype assignments is incremented at step 860. A count of the total number of P′ values that have been evaluated is then incremented at step 862. If step 858 determines that different genotypes have been assigned, then process flow proceeds directly to step 862. The process then determines 864 if there are any SNP positions remaining to be evaluated and if so loops and determines the consistency of genotype assignments for each person for the current SNP being evaluated. The process then determines 866 if there are any SNP positions remaining to be evaluated and if so the process loops until all the SNP positions have been evaluated. Finally the process calculates 868 a measure of the consistency of genotype assignments, by calculating the quotient of the count of the total number of consistently assigned genotypes and the total of the count of the number of genotypes evaluated. This proportion of consistently assigned genotypes can be converted to a percentage if required.

Returning to FIG. 9, the process then determines 820 whether the current proportion of consistent assignments is a maximum and therefore corresponds to a minimum of incorrect assignments. In a first iteration, process 802 simply stores the proportion of consistent assignments and corresponding cut off, as the minimal inconsistent assignments and associated cut off. In subsequent iterations, the process compares the proportion of consistent assignments with the stored current proportion of consistent assignments associated with the minimum and if the current proportion of consistent assignments is greater, then the current proportion and cut off are identified as the minimum and stored. The process then goes over a range of cutoffs in an increasing order. By visual inspection the cutoff with most consistency was selected.

In an alternative embodiment, process 818 counts the number of inconsistent assignments at step 860 and a proportion of inconsistently assigned genotypes is determined at step 868 so that step 820 merely determines the lowest inconsistency proportion and corresponding cutoff. Step 820 therefore determines the cut off value which minimizes the value of the proportion of inconsistently assigned genotypes.

For example, using a first data set obtained using fluorescein as the marker, and varying the cut off between approximately 0.08 and 0.15, in steps of 0.01, a minimum inconsistent genotype assignment of 8.13% was achieved for a cut off of 0.11, in the case in which no seed values were supplied to the EM algorithm. When seed values were supplied to the EM algorithm, then a minimum inconsistent genotype assignment of 5.81% was achieved for a cut off of 0.1. As mentioned supra and as will be described further below, different markers can result in different absolute P′ values and also in different spreads in P′ values. Therefore a scaling factor can be used in analyzing data collected using different markers. For cychrome, a scaling factor of approximately 1.241, relative to fluorescein, has been found to be suitable. Therefore the cut offs used in analyzing cychrome data are scaled by the cychrome scaling factor of 1.241, so that the cut off used in process 402 is approximately 0.1241.

The above described process is predicated on the fact the genotype of an individual does not change when measured in different experiments or using different markers. However, process 802 is based on inconsistencies in assignment, not on the assignments being absolutely accurate. Therefore the process cannot distinguish between consistent inaccurate genotype assignments and consistent accurate genotype assignments. However, as the likelihood of one inaccurate assignment is low the probability of two inaccurate assignments is vanishingly low, and therefore does not significantly affect the reliability of the process. Further, in an inconsistent assignment of genotypes, it is likely that one of the genotype assignments is accurate. Therefore the above mentioned percentages of inconsistent assignments are approximately double the proportion of inaccurate genotypes assignment and so the actual proportion of inaccurate assignments is closer to the 4% and 3% levels respectively. Further, when the chi-squared and Hardy-Weinberg equilibrium tests are applied as confidence checks, the proportion of inconsistently assigned genotypes falls to the approximately 2% level and therefore the number of inaccurate genotype assignments can be considered to be on the order of 1% or less.

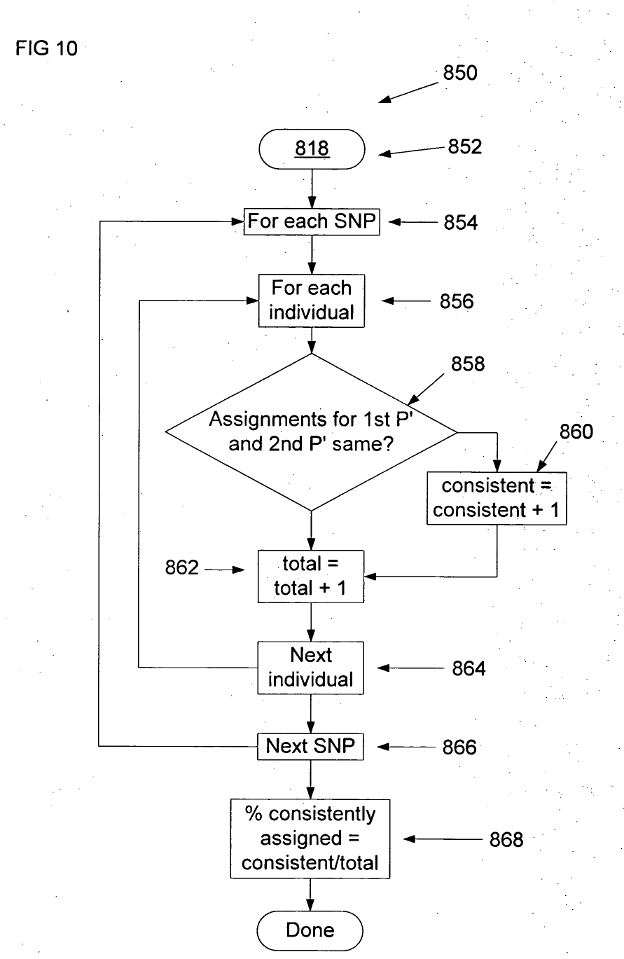

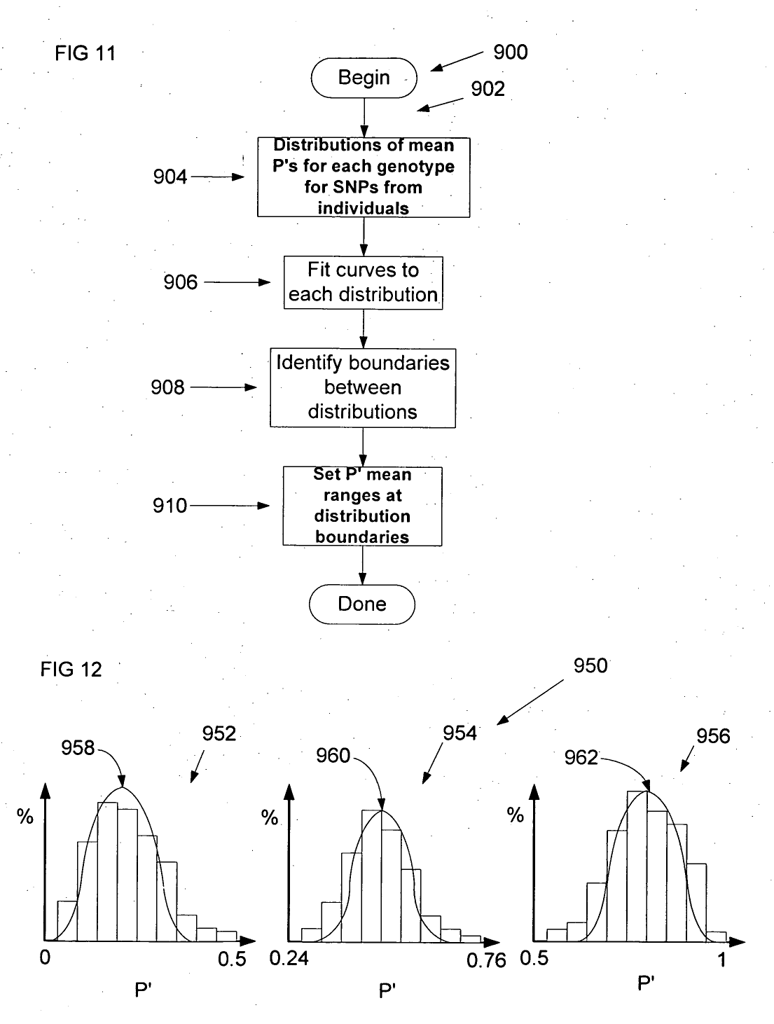

An embodiment of a process for calibrating the range of means of P′ values from which the different genotype bins used in the above described process to assign genotypes when less than three clusters are identified, will now be described with reference to FIGS. 11 and 12. FIG. 11 shows a flow chart 900 illustrating a process 902 for determining ranges of mean P′ values corresponding to a particular genotype for use in step 419 of process 402. FIG. 12 shows graphical representations 950 of histograms of mean P′ values for each of the homozygous alternate 952, heterozygous 954 and homozygous reference 956 genotypes for a few hundred SNP positions and obtained from a group of a few hundred individuals. In general, larger numbers of SNP positions and individuals improve the statistics, although smaller numbers of each can be used.

The process 902 uses a data set of cluster mean P′ values and genotypes assigned to those clusters. The P′ values and genotypes are obtained by applying the EM algorithm to a P′ values for a relatively large number individuals so that three clusters of P′ values can reliably be identified for a SNP. The genotypes can therefore be assigned based on the rank of the clusters. The data set of P′ values used by process 902 is then made up of the P′ values and assigned genotypes for a number of SNPs, in order to provide a large data set so as to improve the statistics.

In a first step 904, the process 902 generates a distribution of mean P′ values for each of the genotypes. Each distribution can be generated by creating data equivalent to a histogram of P′ values for each of the genotypes, as illustrated schematically in FIG. 12. It is not necessary to actually draw histograms. The distributions are visually compared and it is determined where they cross (intersect) regardless of their form (i.e., they are not assumed to be normal).

The process then determines 908 the boundaries between the bins for the three different genotypes by determining the mean P′ value for which adjacent distributions intersect at the same frequency level. For example, the process 908 can determine the mean P′ value for which homozygous alternate and heterozygous distributions have the same magnitude. This mean P′ value defines the boundary between the heterozygous alternate and homozygous bins. Then the heterozygous distribution curve and the homozygous reference distribution curves can be used to determine the mean P′ value falling between the distribution values for which the curves have the same magnitude. This mean P′ value defines the boundary between the homozygous and heterozygous reference bins. For example, for fluorescein, a range of mean P′ values for homozygous alternate of 0 to 0.38, for heterozygous of 0.38 to 0.62, and for homoozygous reference of 0.62 to 1 have been found. For example, for cychrome, a range of mean P′ values for homozygous alternate of 0 to 0.32, for heterozygous of 0.32 to 0.68, and for homozygous reference of 0.68 to 1 have been found. The process then sets the genotype bins 910 as the distribution intersects determined in step 908. The mean P′ value ranges for each genotype and for each marker can then be used in step 419 of process 402 as described previously. It will be appreciated that different markers can have different mean P′ value ranges for each genotype and that suitable values may need to be determined on an ad hoc basis. Further, a greater or lesser number of genotypes will need a correspondingly differing number of ranges of cluster P′ values.

Certain embodiments of the present invention employ processes acting under control of instructions and/or data stored in or transferred through one or more computer systems. Embodiments of the present invention also relate to an apparatus for performing these operations. This apparatus may be specially designed and/or constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium (e.g., optical lines, electrical lines, and/or airwaves).

Examples of program instructions include both machine code, such as produced by a compiler, and files containing higher level code that may be executed by the computer using an interpreter. Further, the program instructions include machine code, source code and any other code that directly or indirectly controls operation of a computing machine in accordance with this invention. The code may specify input, output, calculations, conditionals, branches, iterative loops, etc.

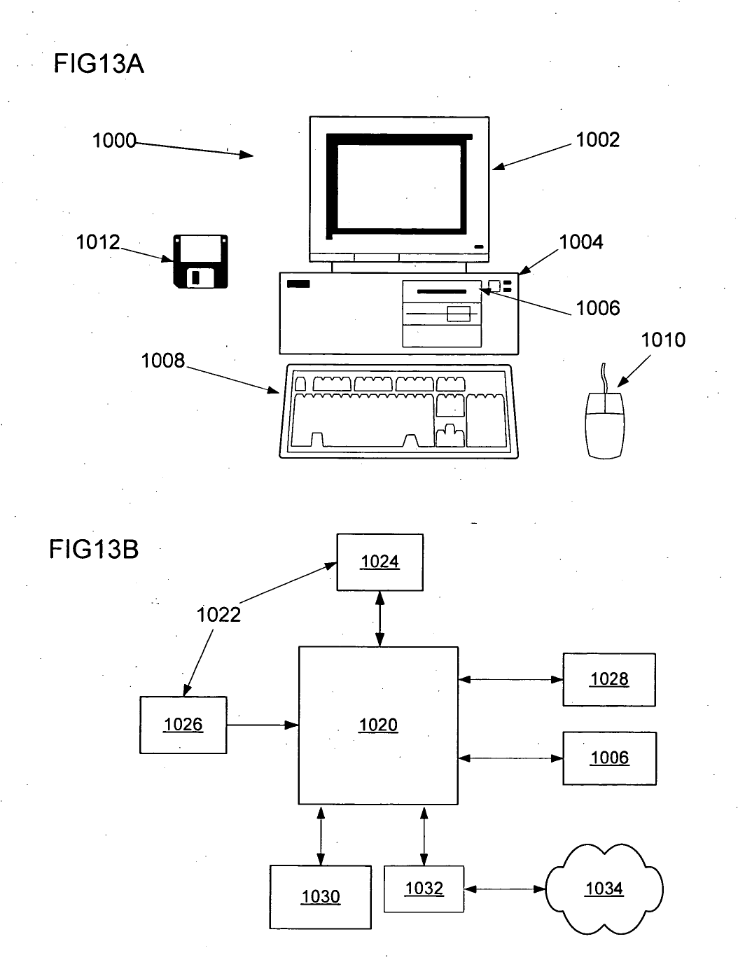

FIGS. 13A and 13B illustrate a computer system 1000 suitable for implementing embodiments of the present invention. FIG. 13A shows one possible physical form of the computer system. Of course, the computer system may have many physical forms ranging from an integrated circuit, a printed circuit board and a small handheld device up to a very large super computer. Computer system 1000 includes a monitor 1002, a housing 1004, a disk drive 1006, a keyboard 1008 and a mouse 1010. Disk 1012 is one example of a computer-readable medium used to transfer data to and from computer system 1000.

FIG. 13B is a block diagram of certain logical components of computer system 1000. Processor(s) 1020 (also referred to as central processing units, or CPU s) are coupled to storage devices including memory 1022. Memory 1022 includes random access memory (RAM) 1024 and read-only memory (ROM) 1026. ROM acts to transfer data and instructions uni-directionally to the CPU and RAM is used typically to transfer data and instructions in a bi-directional manner. Both of these types of memories may include any suitable computer-readable medium, including those described above. A fixed disk 1028 is also coupled bi-directionally to CPU 1020; it provides additional data storage capacity and may also include any of the computer-readable media described below. Fixed disk 1028 may be used to store programs, data and the like and is typically a secondary storage medium (such as a hard disk) that is slower than primary storage. It will be appreciated that the information retained within fixed disk 1028, may, in appropriate cases, be incorporated in standard fashion as virtual memory in memory 1022. Removable disk 1006 may take the form of any of the computer-readable media described below.

CPU 1020 is also coupled to a variety of input/output devices 1030 such as display 1002, keyboard 1008, mouse 1010 and speakers. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 1020 optionally may be coupled to another computer or telecommunications network 1034 using network interface 1032. With such a network interface, it is contemplated that the CPU might receive information from the network, or might output information to the network in the course of performing the above-described method steps. Furthermore, method embodiments of the present invention may execute solely upon CPU 1020 or may execute over a network such as the Internet in conjunction with a remote CPU that shares a portion of the processing.

The flowcharts illustrating the processes carried out can be considered to be merely illustrative of the actual processes carried out, and unless a particular operation or sequence of operations is required by the context or functioning of the invention, then it will be appreciated that some of the steps can be omitted, combined or their sequence altered in line with the general principles described herein. Similarly extension of the general principles taught herein to other genetic markers and organisms and the production of suitable computer programs to implement suitable genotyping methods in line with the general principles underlying the invention will be apparent to persons of ordinary skill in the art in light of the above description.

Although the foregoing invention has been described in some detail for purposes of clarity of understanding, it will be apparent that certain changes and modifications may be practiced within the scope of the appended claims. Therefore, the described embodiments should be taken as illustrative and not restrictive, and the invention should not be limited to the details given herein but should be defined by the following claims and there full scope of equivalents.

What is claimed is:

1. A method for determining a genotype of at least one individual from a genetic marker using at least one measure of the amount of a given allele of the genetic marker in the individual, comprising:
assigning the measure of the amount of the allele to a group using one or more of a probability clustering process and a distance-based clustering process; and
assigning a genotype to the group based on a property of the group, wherein the individual is determined to have the genotype assigned to the group.

2. A method as claimed in claim 1, wherein the method is computer-implemented.

3. A method as claimed in claim 1, wherein the genetic marker is a SNP position.

4. A method as claimed in claim 1, wherein the individual is a haploid organism.

5. A method as claimed in claim 1, wherein the individual is a diploid organism.

6. A method as claimed in claim 5, wherein the diploid organism is a mammal.

7. A method as claimed in claim 6, wherein the mammal is a human.

8. A method as claimed in claim 1, wherein the probability clustering process carries out an expectation maximization algorithm.

9. A method as claimed in claim 1, wherein the distance-based clustering process carries out at least one K-means algorithm.

10. A method as claimed in claim 9, wherein the at least one K-means algorithm is initiated by assigning a plurality of mean values evenly distributed between approximately 0 and approximately 1.

11. A method as claimed in claim 10, wherein 10 mean values are assigned.

12. A method as claimed in claim 9, wherein the at least one K-means algorithm determines a solution for a plurality of subsets of density centers.

13. A method as claimed in claim 12, wherein each subset comprises three density center values.

14. A method as claimed in claim 12, wherein each subset comprises two density center values.

15. A method as claimed in claim 12, wherein each subset comprises one density center value.

16. A method as claimed in claim 12, wherein the plurality of subsets of density center values comprises every combination of subsets of density center values.

17. A method as claimed in claim 9, comprising carrying out a first K-means algorithm and a second K-means algorithm.

18. A method as claimed in claim 17, comprising carrying out a first K-means algorithm and a second K-means algorithm, wherein the first K-means algorithm is initiated by assigning a plurality of mean values; and the second K-means algorithm determines a solution for a plurality of subsets of density centers obtained by the first K-means algorithm.

19. A method as claimed in claim 1, wherein the probability clustering process is initiated using a seed value.

20. A method as claimed in claim 1, wherein the probability clustering process is initiated using a solution obtained by at least one K-means algorithm.

21. A method as claimed in claim 1, wherein a solution obtained by the probability clustering process and/or the distance-based clustering process yields a minimum maximum standard deviation for a distribution of the at least one measure of the amount of the allele.

22. A method as claimed in claim 1, comprising assigning the measure of the amount of the allele using both a probability clustering process and a distance-based clustering process.

23. A method as claimed in claim 22, wherein the probability clustering process carries out an expectation maximization algorithm and the distance-based clustering process carries out a K-means algorithm.

24. A method as claimed in claim 23, wherein the measure of the amount of the allele is assigned to a group based on a comparison between a) a solution determined by the K-means algorithm and b) a solution determined by the expectation-maximization algorithm, wherein the measure of the amount of the allele is assigned to a group according to the solution yielding a minimum maximum standard deviation.

25. A method as claimed in claim 1, wherein the property of the group is a characterizing property of the group and the genotype is assigned based on the characterizing property of the group falling within a one of a plurality of ranges of values of the measure of the amount of the allele, each of the plurality of ranges of values corresponding to a different genotype.

26. A method as claimed in claim 1, wherein the method determines the genotype of a plurality of individuals using a plurality of respective measures of the amount of the allele of the same genetic marker in each of the individuals, and wherein each of the measures of the amount of the allele is assigned to a one of a plurality of potential groups.

27. A method as claimed in claim 26, further comprising;
assessing a confidence of the determinations of the genotypes of the plurality of individuals based on a criterion for p-values corresponding to a particular confidence level.

28. A method as claimed in claim 27, wherein assessing the confidence includes carrying out at least a first and a second evaluation of the confidence in the determinations.

29. A method as claimed in claim 28, wherein the first evaluation comprises a chi-squared distribution to determine the confidence in the determinations.

30. A method as claimed in claim 29, wherein a chi-squared distribution p-value is calculated for each standard deviation of each group from a chi-squared distribution based on a pre-set maximum standard deviation cutoff and a number of degrees of freedom reflecting the number of genotypes in a given group, and the determination of genotypes is rejected if one of the chi-squared distribution p-values does not meet a criterion corresponding to a confidence level.

31. A method as claimed in claim 30, wherein the maximum standard deviation cutoff is set to a value of 0.05.

32. A method as claimed in claim 30, wherein the confidence level is 99.9%.

33. A method as claimed in claim 28, wherein the second evaluation comprises determining a likelihood of the assigned distributions conforming to a corresponding Hardy-Weinberg equilibrium for the plurality of individuals.

34. A method as claimed in claim 33, wherein determining the likelihood includes:
calculating a first sum of a first set of Bayesian factors for all possible permutations of the plurality of individuals over the plurality of potential groups;
calculating a second sum of Bayesian factors from a lowest Bayesian factor in the first set to the Bayesian factor corresponding to the assigned distribution of the individuals between the groups; and
determining a p-value from the quotient of the second sum and the first sum.

35. A method as claimed in claim 34, wherein the determination of genotypes is rejected if the p-value does not meet a criterion corresponding to a confidence level.

36. A method as claimed in claim 35, wherein the confidence level is 99.9%.

37. A method as claimed in claim 28, further comprising determining a ratio between a likelihood of a measure of the amount of an allele corresponding to the assigned group and a likelihood of the measure of the amount of an allele corresponding to the next best fit group.

38. A method as claimed in claim 26, wherein at least one solution from the probability clustering process and the distance-based clustering process includes validating a number of groups to which the measures of the amount of the allele are to be assigned.

39. A method as claimed in claim 38, wherein validating the number of groups includes assigning the measures of the amount of the allele to a first number of groups and using a property of the groups to determine whether the assignment to the first number of groups is reliable.

40. A method as claimed in claim 39, wherein the property of the groups is a mean or median value of each group.

41. A method as claimed in claim 40, wherein using a property of the groups includes determining whether the mean or median values of the groups are sufficiently dissimilar for the groups to constitute different groups.

42. A method as claimed in claim 41, wherein the mean or median values of the groups are considered to be sufficiently dissimilar if they differ by more than a cut off corresponding to a difference which minimizes a number of incorrect genotype assignments for two independent genotype assignments for the same individuals.

43. A method as claimed in claim 39, wherein if it is determined that the assignment to the first number of groups is reliable, then genotypes are assigned to the groups.

44. A method as claimed in claim 39, wherein if it is determined that the assignment to the first number of groups is unreliable, then one or more of the probability clustering process and the distance-based clustering process is repeated to assign the measures of the amount of the allele to a second number of groups different from the first number of groups.

45. A method as claimed in claim 44, wherein the second number of groups is less than the first number of groups.

46. A method as claimed in claim 44, wherein if the measures are assigned to three groups, then the genotype is assigned depending on a ranking of the groups, and if the measures are assigned to less than three groups, then the genotype is assigned depending on a mean value of each group.

47. A method as claimed in claim 46, wherein the genotype is selected from the group consisting of: homozygous reference; homozygous alternate; and heterozygous.

48. A method as claimed in claim 43, wherein the K-means clustering process includes determining a representative value for each group and assigning a genotype to each group based on the respective representative values of each group.

49. A method as claimed in claim 48, wherein assigning the genotype includes determining whether the representative value of a group falls within a one of a plurality of ranges of values.

50. A method as claimed in claim 48 or 49, wherein the representative value is a mean or median of a group.

51. A method as claimed in claim 49, wherein there are three ranges of values and a first range corresponds to a homozygous reference, a second range corresponds to a heterozygous and a third range corresponds to a homozygous alternate.

52. A method as claimed in claim 49, wherein the plurality of ranges of values have been determined by calibrating the measure of the amount of the allele using a sufficiently large sample of individuals to allow groups corresponding to all the different genotypes to be unambiguously determined.

53. A method as claimed in claim 52, wherein at least one boundary of each range is the value at which adjacent corresponding groups intersect.

54. A method as claimed in claim 44, wherein the probability clustering process uses a first set of seed values to assign the first number of groups and a second set of seed values to assign the second number of groups.

55. A method as claimed in claim 54, wherein the seed values are means for respective groups.

56. A method as claimed in claim 55, wherein the first set of seed values comprises approximately 0.2, 0.5 and 0.8, and the second set of seed values comprises approximately 0.3 and 0.7.

57. A method as claimed in claim 55, wherein a ratio of the members of the first set of seed values is approximately 1:1:1 and a ratio of the members of the second set of seed values is approximately 1:1.

58. A computer implemented method for determining one or more genotypes of a plurality of individuals at a SNP position using respective measures of a relative allele amount for the SNP position for each individual, comprising:
assigning the measures of the relative allele amount to a group using one or more of an expectation maximization process and a K-means process;
assigning a genotype to each group identified by the expectation maximization process and/or the K-means process to determine a genotype of each person; and
assessing a confidence of determination of the genotype.

59. A method as claimed in claim 58, wherein the expectation maximization process is initiated using a K-Means algorithm.

60. A method as claimed in claim 58, wherein assessing the confidence includes using a chi-squared distribution to evaluate a spread of at least one of said groups and evaluating whether a distribution of the individuals between the groups conforms to a corresponding Hardy-Weinberg equilibrium distribution.

61. A method as claimed in claim 58, wherein the expectation maximization process determines a number of groups to which to assign the measures and assigns the measures to less than three groups if it is determined that an assignment to three groups would be unreliable.

62. A method as claimed in claim 61, wherein if the measures are assigned to three groups, then the genotype is assigned depending on the rank of the groups, and if the measures are assigned to less than three groups, then the genotype is assigned depending on a mean value of each group.

63. A data processing apparatus for determining the genotype of at least one individual from a genetic marker using at least one measure of the amount of a given allele of the genetic marker in the individual, comprising:
a data processor;
a storage device holding computer readable code in communication with the data processor, the computer readable code including:
computer code which assigns the measure of the amount of the allele to a group by executing one or more of a probability clustering process and a distance-based clustering process; and
computer code which assigns a genotype to the group based on a property of the group and determines the individual to have the genotype assigned to the group.

64. A data processing apparatus as claimed in claim 63, wherein the computer code executing a probability clustering process carries out an expectation maximization algorithm.

65. A data processing apparatus as claimed in claim 63, wherein the computer code executing a distance-based clustering process carries out at least one K-means algorithm.

66. A data processing apparatus as claimed in claim 63, wherein the probability clustering process is initiated using a seed value.

67. A data processing apparatus as claimed in claim 63, wherein the probability clustering process is initiated using a solution obtained by at least one K-means algorithm.

68. A data processing apparatus as claimed in claim 63, wherein a solution obtained by the probability clustering process and/or the distance-based clustering process yields a minimum maximum standard deviation for a distribution of the at least one measure of the amount of the allele.

69. A data processing apparatus as claimed in claim 67, wherein the computer code executes both a probability clustering process and a distance-based clustering process.

70. A data processing apparatus as claimed in claim 69, wherein the probability clustering process carries out an expectation maximization algorithm and the distance-based clustering process carries out at least one K-means algorithm.

71. A data processing apparatus as claimed in claim 70, wherein the computer code assigns the measure of the amount of the allele by comparing a) a solution determined by the K-means algorithm and b) a solution determined by the expectation-maximization solution, wherein the measure of the amount of the allele is assigned to a group according to the solution yielding the minimum maximum standard deviation.

72. A data processing apparatus as claimed in claim 63, further comprising computer code for determining a confidence of the determination of genotype of at least one individual.

73. A computer readable medium comprising computer readable code for determining the genotype of at least one individual from a genetic marker using at least one measure of the amount of an allele of the genetic marker in the individual, and for carrying out the processes of:
assigning the measure of the amount of an allele to a group using one or more of a probability clustering process and a distance-based clustering process; and
assigning a genotype to the group based on a property of the group and determining the individual to have the genotype assigned to the group.

74. A computer readable medium as claimed in claim 73, wherein the probability clustering process comprises an expectation maximization algorithm.

75. A computer readable medium as claimed in claim 73, wherein the distance-based clustering process comprises a K-means algorithm.

76. A computer readable medium as claimed in claim 73, wherein the measure of the amount of the allele is assigned using both a probability clustering process and a distance-based clustering process.

77. A computer readable medium as claimed in claim 73, further comprising computer readable code for determining the confidence of the determination of genotype of at least one individual.

78. A method for calibrating a range of values of measures of the presence of an allele at a genetic marker, comprising:
measuring the value of a measure of the presence of an allele for sufficient individuals to allow groups of individuals corresponding to each expected genotype to be determined, wherein each expected genotype consists of: homozygous reference, homozygous alternate and heterozygous;
determining boundaries between adjacent distributions of values of the measure for each group of individuals; and
assigning a range of values of the measure of the presence of the allele to each genotype, wherein at least one boundary of each range is determined by an intersect of the distributions of adjacent groups.

79. A method for establishing a cut off separation in groups of values of measures of a presence of a given allele at a genetic marker corresponding to different groups for the purposes of carrying out a probability clustering process, comprising:
measuring the values of the presence of the allele at the genetic marker at least twice for the same group of individuals;
assigning the values of the presence of the allele to a number of groups using a clustering algorithm in which the number of groups to which to assign the values is determined using a cut off separation of assigned groups for the independent measurements;
for a cut off value, assigning a genotype to each group for each of the individuals and for each of the independent measurements; and
minimizing a number of inconsistent genotype assignments for the same individual as a function of the cut off.
